(19) United States
(12) Patent Application Publication
(10) Pub. No.: US 2022/0016238 A1
(43) Pub. Date: Jan. 20, 2022

(54) METHODS FOR MAKING MIXED ALLERGEN COMPOSITIONS

(71) Applicant: Alladapt Immunotherapeutics, Inc., Menlo Park, CA (US)

(72) Inventors: Olivia M. Weihe, Redwood City, CA (US); Christopher Cornyn, Woodside, CA (US); Ashley Dombkowski, Woodside, CA (US); Dana McClintock, San Mateo, CA (US)

(21) Appl. No.: 17/298,104

(22) PCT Filed: Nov. 27, 2019
(86) PCT No.: PCT/US2019/063686

§ 371 Date: May 28, 2021

Related U.S. Application Data
(60) Provisional application No. 62/773,643, filed on Nov. 30, 2018.

(51) Int. Cl. A61K 39/35; A23L 3/26

(52) U.S. Cl. CPC A61K 39/35; A61K 2039/577; A23L 3/26

(57) ABSTRACT

Methods of making mixed allergen drug products are provided, wherein the mixed allergen drug products are of known potency and identity and substantially free of replication viable organisms.

### CROSS-REFERENCE TO RELATED APPLICATIONS

This application claims the benefit of and priority to U.S. Provisional Patent Application No. 62/773,643, filed on Nov. 30, 2018, the disclosure of which is hereby incorporated by reference in its entirety for all purposes.

### BACKGROUND

Allergy is a disorder of the immune system characterized by the occurrence of allergic reactions to normally non-pathogenic environmental substances. Allergies are caused by allergens, which may be present in a wide variety of sources including, but not limited to, pollens or other plant components, dust, molds or fungi, foods, additives, latex, transfusion reactions, animal or bird danders, insect venoms, radiocontrast medium, medications or chemicals. Common allergic reactions include eczema, hives, hay fever, and asthma. Mild allergies, like hay fever, are highly prevalent in the human population and cause symptoms such as allergic conjunctivitis, itchiness, and runny nose. In some people, severe allergies to dietary allergens, environmental allergens, or to medication may result in life-threatening anaphylactic reactions if left untreated.

A food allergy is an adverse immune response to a food, for example, a food protein. Common food allergens are found in shellfish, peanuts, tree nuts, fish, milk, eggs, soy and fresh fruits such as strawberries, mangoes, bananas, and apples. Immunoglobulin E (IgE)-mediated food allergies are classified as type-I immediate hypersensitivity reactions. These allergic reactions have an acute onset (as early as seconds) and the accompanying symptoms may include: angioedema (soft tissue swelling of the eyelids, face, lips, tongue, larynx and trachea); hives; itching of the mouth, throat, eyes, or skin; gastrointestinal symptoms such as nausea, vomiting, diarrhea, stomach cramps, or abdominal pain; rhinorrhea or nasal congestion; wheezing; shortness of breath; difficulty swallowing; and anaphylaxis, a severe, whole-body allergic reaction that can result in death. It is estimated that 1 out of 12 children under 21 years of age have a diagnosed food allergy, and over $24 billion is spent per year on health care costs for food allergy reactions, largely due to about 90,000 emergency room visits per year in the U.S. alone for food-induced anaphylaxis. Moreover, deaths occur every year due to fatal food allergies.

Accordingly, there exists a need for allergen compositions that can prevent and/or treat allergies as well as reduce an allergic reaction upon accidental exposure to a food allergen, and methods for making allergen compositions to prevent and/or treat allergies.

### SUMMARY

This disclosure is directed, at least in part, to a method of making a sterile mixed allergen drug product substantially free of replication viable organisms with consistent identity and potency which can be used for oral immunotherapy. For example, in certain embodiments, the method comprises: separately irradiating each of 2 to 20 different raw complete food allergen substances, wherein irradiating comprises applying ionizing radiation to each individual raw complete food allergen substance, thereby producing 2 to 20 individual allergen drug substances each substantially free of replication viable organisms and wherein each individual allergen drug substance retains substantially intact, allergenic proteins; and blending the 2 to 20 individual allergen drug substances together, thereby obtaining the mixed allergen drug product.

In certain embodiments, the disclosure provides a method, wherein the individual raw complete food allergen substances are selected from the group consisting of hazelnut, cashew, pistachio, walnut, pecan, almond, peanut, sesame, soy, hen's egg, bovine milk, wheat, salmon, cod, and shrimp.

In another embodiment, the disclosure provides a method, wherein blending further comprises blending the 2 to 20 individual allergen drug substances with one or more bulking agents and/or pharmaceutically acceptable excipients.

In certain embodiments, the method provides applying ionizing radiation, wherein the ionizing radiation is beta radiation, gamma radiation, alpha radiation, X radiation, or a combination thereof. In some embodiments, the ionizing radiation is applied in one or more doses of about 0.15 kilograys to about 30 kilograys. In certain embodiments, applying ionizing radiation causes a 0.25 to about 0.5° C. per kilogray dose increase in temperature in the raw complete food allergen substance. In further embodiments, the ionizing radiation is produced by a particle emitter having an energy of about 0.5 MeV to about 10 MeV. In embodiments where the ionizing radiation is beta radiation, the beta radiation is single or double sided. In embodiments where the ionizing radiation is gamma radiation, the gamma radiation is produced by cobalt-60 or cesium-137. In embodiments where the ionizing radiation is X radiation, the X radiation is produced using tungsten or tantalum. In certain embodiments, beta radiation is applied at a dose of 5.0, 7.5 or 15 kilograys or more and may be applied once or more than once.

In certain embodiments, the method of the present disclosure further provides milling the mixed allergen drug product to obtain a substantially consistent particle size. In other embodiments, the method further comprises milling one or more than one of the raw complete food allergen substances. In yet other embodiments, the method further comprises milling one or more than one of the individual allergen drug substances.

In other embodiments, the method of the present disclosure further comprises independently packaging each of the 2 to 20 raw complete food allergen substances into separate irradiation compatible packaging before irradiating.

In other embodiments of the present disclosure, each of the 2 to 20 individual allergen drug substances has less than about 1000 CFU/g, less than about 100 CFU/g, or less than about 10 CFU/g of aerobic bacterial organisms. In some embodiments, each of the 2 to 20 individual allergen drug substances has less than about 10 CFU/g of Enterobacteriaceae. In other embodiments, each of the 2 to 20 individual allergen drug substances has less than about 100 CFU/g or less than about 10 CFU/g of yeast and/or mold.

In certain embodiments of the presently disclosed method, each of the 2 to 20 individual allergen drug substances has about 1% to about 10% moisture. In some embodiments, at least one of the 2 to 20 individual allergen drug substances has about 4% to about 7% moisture. In other embodiments, each of the 2 to 20 individual allergen drug substances has about 0.2 to about 0.6 water activity.

In other embodiments of the present disclosure, each individual allergen drug substance has substantially the same protein integrity as compared to a corresponding raw complete food allergen substance, wherein the protein integrity is measured by SDS-PAGE or ELISA.

In certain embodiments, the protein content/potency and/or identity of each raw complete food allergen substance is tested by ELISA.

In other embodiments, each individual allergen drug substance has a substantially similar allergen effect upon administration to a patient as administration of the substantially same protein amount of a corresponding raw complete food allergen substance, wherein allergen effect is measured by immune response in a patient.

In certain embodiments of the method of the present disclosure, the mixed allergen drug product comprises 6 to 20 individual allergen drug substances.

In certain embodiments, the method of the present disclosure provides a mixed allergen drug product comprising about 0.1 mg to about 500 mg, by protein mass, of each individual allergen drug substance. In some embodiments, the mixed allergen drug product comprises 15 or 16 individual allergen drug substances, wherein each individual allergen drug substance is present in about a 2:1 to about 1:2 ratio, by protein weight, with another individual allergen drug substance. In other embodiments, the mixed allergen drug product comprises substantially equal amounts of individual allergen drug substances by total protein weight.

In some embodiments of the present disclosure, the individual allergen drug substances are stable for at least 6 months. In further embodiments, the individual allergen drug substances are stable for at least one year. In other embodiments, the mixed allergen drug product is stable for at least 6 months. In still further embodiments, the mixed allergen drug product is stable for at least one year.

In certain embodiments, the present disclosure provides a method of making a sterile mixed allergen drug product substantially free of replication viable organisms, the method comprising: providing 2 to 20 individual irradiated allergen drug substances each substantially free of replication viable organisms and wherein each individual allergen drug substance retains substantially intact, allergenic proteins; and blending the 2 to 20 individual allergen drug substances together, thereby obtaining the mixed allergen drug product. In another embodiment, the present disclosure provides a method of making a mixed allergen drug product substantially free of replication viable organisms, the method comprising: providing 6 to 20 different raw complete food allergen substances; blending the 6 to 20 different raw complete food allergen substances to produce a bulk substance; and irradiating the bulk substance with ionizing radiation, thereby obtaining the mixed allergen drug product.

Also disclosed is a mixed allergen drug product that is substantially free of replication viable organisms prepared by any one of the methods disclosed herein. Also contemplated is a mixed allergen drug product prepared by any one of the methods disclosed herein for oral immunotherapeutic treatment of food allergy in a child or adult. In another embodiment, a mixed allergen drug product as disclosed herein is for mixture with a food to which the child or adult is not allergic.

In another embodiment, the present disclosure provides a method of making a sterile allergen drug product substantially free of replication viable organisms, the method comprising: irradiating a raw complete food allergen substance, wherein irradiating comprises applying ionizing radiation to the raw complete food allergen substance, thereby producing an individual allergen drug substance substantially free of replication viable organisms, and wherein each allergen drug substance retains substantially intact, allergenic proteins. In a certain embodiment, the raw complete food allergen substance is selected from the group consisting of hazelnut, cashew, pistachio, walnut, pecan , almond, peanut, sesame, soy, hen's egg, bovine milk, wheat, salmon, cod, and shrimp.

### BRIEF DESCRIPTION OF THE DRAWINGS

FIG. 1A shows an SDS-PAGE of non-irradiated and beta-radiated (E-beam irradiated) hazelnut powder. Lane 1=protein standard ladder; lane 2=E-beam irradiated hazelnut powder (7.5 kGy); lane 3=non-irradiated hazelnut powder. FIG. 1B shows optical densitometry of the SDS-PAGE of FIG. 1A.

FIG. 2A shows an SDS-PAGE of non-irradiated and beta-radiated (E-beam irradiated) cashew powder. Lane 1=protein standard ladder; lane 2=E-beam irradiated cashew powder (7.5 kGy); lane 3=non-irradiated cashew powder. FIG. 2B shows optical densitometry of the SDS-PAGE of FIG. 2A.

FIG. 3A shows an SDS-PAGE of non-irradiated and beta-radiated (E-beam irradiated) pistachio powder. Lanes 1 and 4=protein standard ladder; lane 2=E-beam irradiated pistachio powder (7.5 kGy); lane 3=non-irradiated pistachio powder. FIG. 3B shows optical densitometry of the SDS-PAGE of FIG. 3A.

FIG. 4A shows an SDS-PAGE of non-irradiated and beta-radiated (E-beam irradiated) walnut powder. Lanes 1 and 4=protein standard ladder; lane 2=E-beam irradiated walnut powder (7.5 kGy); lane 3=non-irradiated walnut powder. FIG. 4B shows optical densitometry of the SDS-PAGE of FIG. 4A.

FIG. 5A shows an SDS-PAGE of non-irradiated and beta-radiated (E-beam irradiated) pecan powder. Lanes 1 and 4=protein standard ladder; lane 2=E-beam irradiated pecan powder (7.5 kGy); lane 3=non-irradiated pecan powder. FIG. 5B shows optical densitometry of the SDS-PAGE of FIG. 5A.

FIG. 6A shows an SDS-PAGE of non-irradiated and beta-radiated (E-beam irradiated) almond powder. Lane 1=protein standard ladder; lane 2=E-beam irradiated almond powder (7.5 kGy); lane 3=non-irradiated almond powder. FIG. 6B shows optical densitometry of the SDS-PAGE of FIG. 6A.

FIG. 7A shows an SDS-PAGE of non-irradiated and beta-radiated (E-beam irradiated) peanut powder. Lane 1=protein standard ladder; lane 2=E-beam irradiated peanut powder (7.5 kGy); lane 3=non-irradiated peanut powder. FIG. 7B shows optical densitometry of the SDS-PAGE of FIG. 7A.

FIG. 8A shows an SDS-PAGE of non-irradiated and beta-radiated (E-beam irradiated) sesame powder. Lane 1=protein standard ladder; lane 2=E-beam irradiated sesame powder (7.5 kGy); lane 3=non-irradiated sesame powder. FIG. 8B shows optical densitometry of the SDS-PAGE of FIG. 8A.

FIG. 9A shows an SDS-PAGE of non-irradiated and beta-radiated (E-beam irradiated) soy protein powder. Lanes 1 and 4=protein standard ladder; lane 2=E-beam irradiated soy protein powder (7.5 kGy); lane 3=non-irradiated soy protein powder. FIG. 9B shows optical densitometry of the SDS-PAGE of FIG. 9A.

FIG. 10A is an SDS-PAGE of non-irradiated and E-beam irradiated hen's egg powder. Lane 1=protein standard ladder; lanes 2 and 3=non-irradiated hen's egg powder; lanes 4 and 5=E-beam irradiated hen's egg powder (7.5 kGy); and lanes 6 and 7=E-beam irradiated hen's egg powder (15 kGy). FIG. 10B shows optical densitometry of the SDS-PAGE of FIG. 10A.

FIG. 11A is an SDS-PAGE of non-irradiated and beta-radiated (E-beam irradiated) bovine milk protein isolate. Lanes 1 and 8=protein standard ladder; lanes 2 and 3=non-irradiated bovine milk protein isolate; lanes 4 and 5=E-beam irradiated bovine milk protein isolate (7.5 kGy); and lanes 6 and 7=E-beam irradiated bovine milk protein isolate (15 kGy). FIG. 11B shows optical densitometry of the SDS-PAGE of FIG. 11A.

FIG. 12A shows an SDS-PAGE of non-irradiated and beta-radiated (E-beam irradiated) wheat protein powder. Lanes 1 and 4=protein standard ladder; lane 2=E-beam irradiated wheat protein powder (7.5 kGy); lane 3=non-irradiated soy powder. FIG. 12B shows optical densitometry of the SDS-PAGE of FIG. 12A.

FIG. 13A shows an SDS-PAGE of non-irradiated and beta-radiated (E-beam irradiated) salmon protein powder. Lane 1=protein standard ladder; lane 2=E-beam irradiated salmon protein powder (7.5 kGy); lane 3=non-irradiated salmon protein powder. FIG. 13B shows optical densitometry of the SDS-PAGE of FIG. 13A.

FIG. 14A shows an SDS-PAGE of non-irradiated and beta-radiated (E-beam irradiated) cod powder. Lane 1=protein standard ladder; lane 2=E-beam irradiated cod powder (7.5 kGy); lane 3=non-irradiated cod powder. FIG. 14B shows optical densitometry of the SDS-PAGE of FIG. 14A.

FIG. 15A shows an SDS-PAGE of non-irradiated and beta-radiated (E-beam irradiated) shrimp protein powder. Lanes 1 and 4=protein standard ladder; lane 2=E-beam irradiated shrimp protein powder (7.5 kGy); lane 3=non-irradiated shrimp protein powder. FIG. 15B shows optical densitometry of the SDS-PAGE of FIG. 15A.

FIG. 16 is a line graph showing the particle size distribution of non-irradiated hazelnut powder (open circles), and E-beam irradiated hazelnut powder (7.5 kGy, closed triangles).

FIG. 17 is a line graph showing the particle size distribution of non-irradiated cashew powder (open circles), and E-beam irradiated cashew powder (7.5 kGy, closed triangles).

FIG. 18 is a line graph showing the particle size distribution of non-irradiated pistachio powder (open circles), and E-beam irradiated pistachio powder (7.5 kGy, closed triangles).

FIG. 19 is a line graph showing the particle size distribution of non-irradiated walnut powder (open circles), and E-beam irradiated walnut powder (7.5 kGy, closed triangles).

FIG. 20 is a line graph showing the particle size distribution of non-irradiated pecan powder (open circles), and E-beam irradiated pecan powder (7.5 kGy, closed triangles).

FIG. 21 is a line graph showing the particle size distribution of non-irradiated almond powder (open circles), and E-beam irradiated almond powder (7.5 kGy, closed triangles).

FIG. 22 is a line graph showing the particle size distribution of non-irradiated peanut powder (open circles), and E-beam irradiated peanut powder (7.5 kGy, closed triangles).

FIG. 23 is a line graph showing the particle size distribution of non-irradiated sesame powder (open circles), and E-beam irradiated sesame powder (7.5 kGy, closed triangles).

FIG. 24 is a line graph showing the particle size distribution of non-irradiated soy protein powder (open circles), and E-beam irradiated soy protein powder (7.5 kGy, closed triangles).

FIG. 25 is a line graph showing the particle size distribution of non-irradiated hen's egg powder (open circles), E-beam irradiated hen's egg powder (7.5 kGy, closed triangles), and E-beam irradiated hen's egg powder (15 kGy, closed squares).

FIG. 26 is a line graph showing the particle size distribution of non-irradiated bovine milk protein isolate (open circles), E-beam irradiated bovine milk protein isolate (7.5 kGy, closed triangles), and E-beam irradiated bovine milk protein isolate (15 kGy, closed squares).

FIG. 27 is a line graph showing the particle size distribution of non-irradiated wheat protein powder (open circles), and E-beam irradiated wheat protein powder (7.5 kGy, closed triangles).

FIG. 28 is a line graph showing the particle size distribution of non-irradiated salmon protein powder (open circles), and E-beam irradiated salmon protein powder (7.5 kGy, closed triangles).

FIG. 29 is a line graph showing the particle size distribution of non-irradiated cod powder (open circles), and E-beam irradiated cod powder (7.5 kGy, closed triangles).

FIG. 30 is a line graph showing the particle size distribution of non-irradiated shrimp protein powder (open circles), and E-beam irradiated shrimp protein powder (7.5 kGy, closed triangles).

FIG. 31 is a flow diagram showing a manufacturing process for the production of clinical-grade mixed allergen drug product.

FIG. 32 is a line graph showing a representative ELISA curve of an almond powder drug substance compared to an almond powder reference standard, non-specific food allergen substance (shrimp powder), and excipient control (isomalt).

### DETAILED DESCRIPTION

Disclosed herein are methods of making a sterile mixed allergen drug product substantially free of replication viable organisms.

As used herein, “raw complete food allergen substances” refer to food substances containing all possible antigenic components (for example, allergenic proteins). Raw complete food allergen substances may include, but are not limited to, unprocessed or processed food substances, concentrated food substances, and isolated food substances.

“Allergenic proteins”, as used herein, are antigenic components of food allergen substances that are, either directly or indirectly, responsible for eliciting a biological allergenic response when administered to a patient. Allergenic proteins may include, but are not limited to, nut proteins such as hazelnut proteins (e.g., Cor a 1, Cor a 2, Cor a 6, Cor a 8, Cor a 9, Cor a 10, Cor a 11, Cor a 12, Cor a 13, and Cor a 14), cashew proteins (e.g., Ana o 1, Ana o 2, and Ana o 3), pistachio proteins (e.g., Pis v 1, Pis v 2, Pis v 3, Pis v 4, and Pis v 5), walnut proteins (e.g., Jug r 1, Jug r 2, Jug r 3, Jug r 4, Jug r 5, Jug r 6, Jug r 7, and Jug r 8, Jug n1, Jug n 2, and Jug n 4), pecan proteins (e.g., Car i 1, Car i 2, and Car i 4), almond proteins (e.g., Pm du 3, Pm du 4, Pm du 5, Pm du 6, and Pm du 8), and peanut proteins (e.g., Ara h 1, Ara h 2, Ara h 3, Ara h 4, Ara h 5, Ara h 6, Ara h 7, Ara h 8, Ara h 9, Ara h 10, Ara h 11, Ara h 12, Ara h 13, Ara h 14, Ara h 15, Ara h 16, and Ara h 17). Allergenic proteins may also include, but are not limited to, animal proteins such as egg proteins (e.g., Gal d 1, Gal d 2, Gal d 3, Gal d 4, Gal d 5, Gal d 6, Gal d 7, Gal d 8, Gal d 9, Gal d 10), milk proteins (e.g., Bos d 2, Bos d 3, Bos d 4, Bos d 5, Bos d, 6, Bos d 7, Bos d 8, Bos d 9, Bos d 10, Bos d 11, and Bos d 12), salmon proteins (e.g., Onc k 5, Sal s 1, Sal s 2, and Sal s 3), cod proteins (e.g., pGad c 1, Gad m 1, Gad m 2, and Gad m 3), and shrimp proteins (e.g., Cra c 1, Cra c 2, Cra c 4, Cra c 5, Cra c 6, Cra c 8, Lit v 1, Lit v 2, Lig v 3, Lit v 4, Mete 1, Pan b 1, Pen a 1, Pen i 1, Pen m 1, Pen m 2, Pen m 3, Pen m 4, and Pen m 6). Allergenic proteins may further include, but are not limited to, non-nut plant proteins such as wheat proteins (e.g., Tri a 12, Tri a 14, Tri a 15, Tri a 17, Tri a 18, Tri a 19, Tri a 20, Tri a 21, Tri a 25, Tri a 26, Tri a 27, Tri a 28, Tri a 29, Tri a 30, Tri a 31, Tri a 32, Tri a 33, Tri a 34, Tri a 35, Tri a 36, Tri a 37, Tri a 39, Tri a 40, Tri a 41, Tri a 42, Tri a 43, Tri a 44, and Tri a 45), soy proteins (e.g., Gly m 1, Gly m 1.0101, Gly m 2, Gly m 3, Gly m 4, Gly m 5, Gly m 6, Gly m 7, and Gly m 8), sesame seed proteins (e.g., Ses i 1, Ses i 2, Ses i 3, Ses i 4, Ses i 5, Ses i 6, and Ses i 7), kiwi proteins (e.g., Act c 1, Act c 5, Act c 8, Act c 10, Act d 1, Act d 2, Act d 3, Act d 4, Act d 5, Act d 6, Act d 7, Act d 8, Act d 9, Act d 10, Act d 11, Act d 12, and Act d 13), carrot proteins (e.g., Dau c 1, Dau c 4, and Dau c 5), celery proteins (e.g., Api q 1, Api q 2, Api q 3, Api q 4, Api q 5, and Api q 6), stone fruit proteins (e.g., Pm ar 1, Pm ar 3, Pm av 1, Pm av 2, Pm av 3, Pm av 4, Pm p 1, Pm p 2, Pm p 3, Pm p 4, Pm p 7, and Pm d 3), and oat proteins.

The term “ionizing radiation” refers to radiation having sufficient energy to remove electrons from atoms or molecules, thereby ionizing them. In the context of the present disclosure, “ionizing radiation” particularly refers to radiation having sufficient energy to ionize and disrupt the DNA of microorganisms.

As used herein, “individual allergen drug substances” refers to complete food allergen substances that have been subjected to a sufficient dose or doses of ionizing radiation to be rendered substantially free of replication viable organisms.

By “replication viable organisms”, it is meant organisms that are capable of multiplying/reproducing/propagating and producing colony forming units (CFU) on a plate culture.

Presently disclosed, for example, is a method of making a sterile mixed allergen drug product substantially free of replication viable organisms, the method comprising: separately irradiating each of 2 to 20 different raw complete food allergen substances, wherein irradiating comprises applying ionizing radiation to each individual raw complete food allergen substance, thereby producing 2 to 20 individual allergen drug substances, each substantially free of replication viable organisms and wherein each individual allergen drug substance retains substantially intact, allergenic proteins; and blending the 2 to 20 individual allergen drug substances together, thereby obtaining the mixed allergen drug product.

In certain embodiments, a disclosed method comprises separately irradiating each of 2 to 20, for example, 4 to 20, 6 to 20, 8 to 20, 10 to 20, 12 to 20, 14 to 20, 16 to 20, 18 to 20, 2 to 18, 4 to 18, 6 to 18, 8 to 18, 10 to 18, 12 to 18, 14 to 18, 16 to 18, 2 to 16, 4 to 16, 6 to 16, 8 to 16, 10 to 16, 12 to 16, or 14 to 16 different raw complete food allergen substances. For example, 2, 3, 4, 5, 6, 7, 8, 9, 10, 11, 12, 13, 14, 15, 16, 17, 18, 19, or 20 different raw complete food allergen substances are irradiated. In certain embodiments, 2 different raw complete food allergen substances are irradiated. In a particular embodiment, 15 or 16 different raw complete food allergen substances are irradiated. It will be appreciated that two or more raw complete food allergen substances may be in combination prior to irradiation. For example, 4 to 20, 8 to 20, 10 to 20, 12 to 20, 14 to 20, 16 to 20, 18 to 20, 2 to 18, 4 to 18, 6 to 18, 8 to 18, 10 to 18, 12 to 18, 14 to 18, 16 to 18, 2 to 16, 4 to 16, 6 to 16, 8 to 16, 10 to 16, 12 to 16, or 14 to 16 different raw complete food allergen substances may be combined prior to irradiation. In a further example, 2, 3, 4, 5, 6, 7, 8, 9, 10, 11, 12, 13, 14, 15, 16, 17, 18, 19, or 20 different raw complete food allergen substances may be combined prior to irradiation.

A method of the present disclosure may include separately irradiating any of the raw complete allergen drug substances described herein. For example, in certain embodiments, the raw complete food allergen substances are selected from a group consisting of nut, seed, legume, egg, dairy, grain, fish and crustacean. In particular embodiments, the raw complete food allergen substances are selected from the group consisting of hazelnut, cashew, pistachio, walnut, pecan, almond, peanut, sesame, soy, hen's egg, bovine milk, wheat, salmon, cod, and shrimp. In certain embodiments, the raw complete allergen drug substances are hazelnut, cashew, pistachio, walnut, pecan, almond, peanut, sesame, soy, egg, milk, wheat, salmon, cod, and shrimp. In other embodiments, the raw complete allergen drug substances are egg and milk. It will be appreciated that the individual raw complete food allergen substances contemplated herein may each be present as a meal, flour, powder, and/or protein concentrate.

As contemplated in the present disclosure, separately irradiating each of 2 to 20 different raw complete food allergen substances (e.g., 4 to 20, 6 to 20, 8 to 20, 10 to 20, 12 to 20, 14 to 20, 16 to 20, 18 to 20, 2 to 18, 4 to 18, 6 to 18, 8 to 18, 10 to 18, 12 to 18, 14 to 18, 16 to 18, 2 to 16, 4 to 16, 6 to 16, 8 to 16, 10 to 16, 12 to 16, or 14 to 16 raw complete food allergen substances) comprises applying ionizing radiation to each individual raw complete food allergen substance. In certain embodiments, applying ionizing radiation comprises applying beta radiation, also referred to as electron radiation or E-beam radiation. For example, the beta radiation is applied as single or double sided. In other embodiments of the present disclosure, applying ionizing radiation comprises applying gamma radiation, for example, produced by cobalt-60 or cesium-137. In other embodiments, applying ionizing radiation comprises applying alpha radiation. In another embodiment, applying ionizing radiation comprises applying X radiation, for example, produced using tungsten or tantalum. In yet another embodiment, any two or more ionizing radiations selected from the group consisting of beta radiation, gamma radiation, alpha radiation, and X radiation may be applied in combination to the 2 to 20 different raw complete food allergen substances.

In certain embodiments, the ionizing radiation applied to each of the 2 to 20 different raw complete food allergen substances is produced by a particle emitter having an energy of about 0.5 MeV to about 10 MeV.

Further contemplated methods disclosed herein may include separately irradiating each of 2 to 20 different raw complete food allergen substances to render any microorgansims on or in the individual raw complete food allergen substances replication inviable. Radiation doses contemplated in the present disclosure are about 0.15 kilograys to about 30 kilograys. For example, in a particular embodiment, the ionizing radiation is beta radiation applied at a dose of 5.0 kGy, 7.5 kGy, 15 kGy, or more. In another embodiment, the ionizing radiation is beta radiation applied once or more than once. It will be appreciated that such contemplated doses are sufficient to render any microorganisms on or in the individual raw complete food allergen substances replication inviable and are within the set maximum allowable dosages for food irradiation applications set by the United States Federal Drug Administration. Furthermore, it will be appreciated that application of ionizing radiation causes about a 0.25 to about 0.5° C. increase in temperature per kilogray dose of radiation in the raw complete food allergen sub stance.

In certain embodiments, each of the 2 to 20 raw complete food allergen substances are independently packaged in irradiation compatible packaging before applying ionizing radiation. “Irradiation compatible” is understood to mean that applying the same ionizing radiation to the packaging material as concurrently applied to each of the individual raw complete food allergen substances packaged therein, does not cause changes in the packaging material that affect its integrity and functionality as a barrier to chemical or microbial contamination. Furthermore, “irradiation compatible” is understood to mean that exposure to ionizing radiation does not alter the packaging to cause a chemical in the packaging to be added to the individual raw complete food allergen substances packaged therein. For example, each of the 2 to 20 raw complete food allergen substances may be packaged in irradiation compatible packaging, wherein 10 kGy of ionizing radiation is concurrently applied to the irradiation compatible packaging and the individual raw complete food allergen substance packaged therein. It is further contemplated that sterility of each individual allergen drug substance following application of ionizing radiation is preserved as long as each individual allergen drug substance remains packaged in the irradiation compatible packaging and the integrity of the irradiation compatible packaging is uncompromi sed.

In the methods of the present disclosure, separately irradiating each of 2 to 20 different raw complete food allergen substances with ionizing radiation produces 2 to 20 individual allergen drug substances that are each substantially free of replication viable organisms. In certain embodiments, individual allergen drug substances of the present disclosure are substantially free of replication viable bacteria, yeast, and/or molds. For example, each of the 2 to 20 individual allergen drug substances has less than about 1000 CFU/g, less than about 100 CFU/g, or less than about 10 CFU/g of aerobic bacterial organisms. In another example, each of the 2 to 20 individual allergen drug substances has less than about 10 CFU/g of Enterobacteriaceae. In yet another example, each of the 2 to 20 individual allergen drug substances has less than about 100 CFU/g or less than about 10 CFU/g of yeast. In another example, each of the 2 to 20 individual allergen drug substances has less than about 100 CFU/g or less than about 10 CFU/g of mold.

It is contemplated that applying ionizing radiation to each of the 2 to 20 different raw complete food allergen substances will not substantially alter protein integrity. For example, in certain embodiments, each individual allergen drug substance has substantially the same protein integrity as compared to a corresponding raw complete food allergen substance as measured by SDS-PAGE. In further embodiments, it is contemplated that the protein content, potency, and/or identity of each raw complete food allergen substance and/or each individual allergen drug substance is tested by ELISA and/or lateral flow assay. As used herein, “potency” refers to the ability of a raw complete food allergen substance or individual allergen drug substance to react with an antibody having binding specificity to the raw complete food allergen substance or individual allergen drug substance. In some embodiments, potency can be quantified so as to provide consistent concentrations of individual allergen drug substances in a mixed allergen drug product during clinical trials, as well as beyond during commercialization of the drug product. As used herein, “lateral flow assay” refers to an immunochromatographic assay used to detect the presence of a raw complete food allergen substance or individual allergen drug substance in a sample.

It is also contemplated that applying ionizing radiation to each of the 2 to 20 different raw complete food allergen substances will not substantially affect the ability of each individual allergen drug substance to elicit an allergen effect upon administration to a patient. In certain embodiments, each individual allergen drug substance has a substantially similar allergen effect upon administration to a patient as administration of the substantially same protein amount of a corresponding raw complete food allergen substance. In certain embodiments, the allergen effect is measured by the immune response in the patient, for example, measuring the production of IgE or cytokines, or measuring immune cell activation in response to administration of each individual allergen drug substance. In other embodiments, the allergen effect is measured by the immune response in vitro, for example, measuring the production of IgE or cytokines after activation of immune cells, or measuring activation of immune cell cultures.

In certain embodiments, each of the 2 to 20 irradiated individual allergen drug substances has about 1% to about 10% moisture. For example, at least one of the 2 to 20 individual allergen drug substances may have about 4% to about 7% moisture. In another example, each of the 2 to 20 irradiated individual allergen drug substances has about 4% to about 7% moisture. In other embodiments, each of the 2 to 20 irradiated individual allergen drug substances has about 0.2 to about 0.6 water activity. “Water activity” is understood as the ratio between the vapor pressure of each of the individual allergen drug substances, and the vapor pressure of distilled water under identical conditions. It will be appreciated that water activity is a measure of the water that is not bound to the molecules of each of the individual allergen drug substances and thus capable of supporting growth of bacteria, yeast and mold. Furthermore, it will be appreciated that water activity may be measured using suitable electronic instruments such as moisture meters, moisture-humidity meters, hygrometers, and relative humidity systems.

It is contemplated that each of the 2 to 20 individual allergen drug substances are stable for at least 1 week, 2 weeks, 1 month, 2 months, 3 months, 4 months, 5 months, 6 months, one year, 2 years, 5 years, or more. It is further contemplated that the mixed allergen drug product is stable for at least 1 week, 2 weeks, 1 month, 2 months, 3 months, 4 months, 5 months, 6 months, one year, 2 years, 5 years, or more.

In other embodiments, each of the 2 to 20 different raw complete food allergen substances are provided as 2 to 20 individual irradiated allergen drug substances substantially free of replication viable organisms and wherein each individual allergen drug substance retains substantially intact allergenic proteins.

In certain embodiments, a presently disclosed method of making the sterile mixed allergen drug product further comprises blending the 2 to 20 individual allergen drug substances together. In a particular embodiment, a presently disclosed method further comprises blending 6 to 20 individual allergen drug substances. The amount of each individual allergen drug substance in the sterile mixed allergen drug product may vary as desired. In certain embodiments, the mixed allergen drug product comprises about 0.1 mg to about 500 mg, by protein mass, of each individual allergen drug substance. In yet further embodiments, the mixed allergen drug product comprises 15 or 16 individual allergen drug substances, wherein each individual allergen drug substance is present in about a 2:1 to about 1:2 ratio, by protein weight, with another individual allergen drug substance. For example, the mixed allergen drug product may comprise substantially equal amounts of individual allergen drug substances by total protein weight.

In a particular embodiment, a presently disclosed method comprises a mixed allergen drug product comprising individual allergen drug substances selected from the group consisting of hazelnut, cashew, pistachio, walnut, pecan, almond, peanut, sesame, soy, hen's egg, bovine milk, wheat, salmon, cod, and shrimp. It will be appreciated that the individual allergen drug substances contemplated herein may each be present as a meal, flour, powder, and/or protein concentrate.

In certain embodiments, a method of the present disclosure further comprises blending the 2 to 20 individual allergen drug substances with one or more bulking agents. Contemplated bulking agents may include any bulking agent described herein. In certain embodiments, the bulking agent comprises a sugar or sugar alcohol, for example, sucrose, maltodextrin, trehalose, trehalose dehydrate, mannitol, lactose, dextrose, fructose, raffinose, aldose, ketose, glucose, sucrose, xylitol, sorbitol, isomalt, erythritol, pentitol, hexitol, malitol, aceculfame potassium, talin, glycyrrhizin, sucralose, aspartame, saccharin, sodium saccharin, maltodextrin, neohesperidin dihydrochalcone, monoammonium glycyrrhizinate, sodium cyclamate, or any combination thereof. In certain embodiments, the bulking agent comprises maltodextrin, or sucrose, or a combination thereof. In certain embodiments, the bulking agent comprises maltodextrin and sucrose at a weight ratio of about 3:1. Without wishing to be bound by theory, it is believed that bulking agents reduce the fat content of a mixed allergen drug product to aid in downstream processing (e.g., milling).

In certain embodiments, the methods disclosed in the present disclosure may include blending the 2 to 20 individual allergen drug substances with a pharmaceutically acceptable excipient. Contemplated excipients may include any pharmaceutically acceptable excipient described herein. In certain embodiments, the pharmaceutically acceptable excipient comprises, for example, a food safe oil, a polysaccharide (for example, gellan gum), flavoring, a food safe salt (for example, dipotassium phosphate), and/or natural compounds (for example, vanilla extract or cinnamon).

In another embodiment, the disclosure provides a method of making a mixed allergen drug product substantially free of replication viable organisms, the method comprising: providing 2 to 20 different raw complete food allergen substances; blending the 2 to 20 raw complete food allergen substances to produce a bulk substance; and irradiating the bulk substance with ionizing radiation, thereby producing the mixed allergen drug product substantially free of replication viable organisms and wherein the mixed allergen drug product retains substantially intact allergenic proteins.

In certain embodiments, a contemplated method disclosed herein further comprises milling the mixed allergen drug product, for example, in a conical mill. The milling may, for example, comprise using a rotor speed of about 9000 RPM, or may further comprise applying a vacuum suction through the conical mill. The milling may, for example, comprise passing the mixed allergen drug product through a screen with an opening size of about 0.033 inches. Without wishing to be bound by theory, it is believed that milling reduces grittiness and large particle size and increases blend homogeneity.

Also contemplated is a method of making a mixed allergen drug product, wherein the mixed allergen drug product is further mixed with a physiologically acceptable delivery vehicle to produce a physiologically acceptable composition. Mixed allergen drug products can be further incorporated into a variety of formulations for administration to a subject. More particularly, a mixed allergen drug product can be formulated into a physiological acceptable composition by combination with appropriate, physiologically acceptable carriers or diluents, for example, a vegetable oil. In certain embodiments, a disclosed mixed allergen drug product is designed for oral immunotherapeutic treatment of food allergy in a child or adult, for example, as dispersible powders or granules, foods, tablets, troches, lozenges, emulsions, etc. Compositions intended for oral use may be prepared according to any convenient protocol for the manufacture of pharmaceutical compositions and such compositions may contain one or more agents selected from the group consisting of sweetening agents (e.g., glycerol, propylene glycol, sorbitol, or sucrose), flavoring agents, coloring agents and preserving agents in order to provide palatable preparations.

Also contemplated is a method of making a mixed allergen drug product, wherein the mixed allergen drug product is mixed with food to which a child or adult is not allergic. For example, foods may include, but are not limited to: baby or infant formula, baby food (e.g., pureed food suitable for infant or toddler consumption), chips, cookies, breads, spreads, creams, yogurts, liquid drinks, chocolate containing products, candies, ice creams, cereals, coffees, pureed food products, etc.

In yet another embodiment, the present disclosure provides a method of making a sterile allergen drug product substantially free of replication viable organisms, the method comprising: irradiating a raw complete food allergen substance, wherein irradiating comprises applying ionizing radiation to the raw complete food allergen substance, thereby producing the sterile allergen drug product substantially free of replication viable organisms and wherein the sterile allergen drug product retains substantially intact, allergenic proteins. For example, the raw complete food allergen substance is selected from the group consisting of hazelnut, cashew, pistachio, walnut, pecan, almond, peanut, sesame, soy, hen's egg, bovine milk, wheat, salmon, cod, and shrimp.

Throughout the description, where apparatus, devices, and systems are described as having, including, or comprising specific components, or where processes and methods are described as having, including, or comprising specific steps, it is contemplated that, additionally, there are apparatus, devices, and systems that consist essentially of, or consist of, the recited components, and that there are processes and methods that consist essentially of, or consist of, the recited processing steps.

The foregoing examples are presented herein for illustrative purposes only, and should not be construed as limiting in any way.

### EXAMPLES

### Example 1

A series of representative tests can establish the identity, strength, quality, and purity of each of 15 different raw complete food allergen substances (e.g., hazelnut, cashew, pistachio, walnut, pecan, almond, peanut, sesame, soy, hen's egg, bovine milk, wheat, salmon, cod, and shrimp) used to produce an exemplary dry powder mixed allergen drug product (see TABLE 1).

For each individual raw complete food allergen, the macroscopic appearance (e.g., powder granularity, color) was documented and compared to corresponding raw complete food allergen standards.

TABLE 1
Summary of Assays for Analyzing Food Allergen
Substances and Drug Allergen Substances

| Purpose | Test | Acceptance Criteria |
|---|---|---|
| General | Appearance (powder/color) | Conforms to expected |
| | | appearance |
| General | Residual Moisture | |
| Identity/Potency | ELISA and Lateral Flow | Positive Identity |
| | Assay | |
| Identity/Purity | SDS-PAGE | Banding pattern |
| | | substantially similar to |
| | | the reference material. |
| Strength | Total protein content | |
| | (for e.g. Lowry) | |

Residual moisture was determined for each individual raw complete food allergen using Trimetric method/Azeotropic method/Gravimetric method (see Example 4).

Total extractable protein content of each individual raw complete food allergen and individual allergen drug substance was measured by Lowry protein assay. Extraction efficiencies were calculated by comparing the expected theoretical concentration (as determined by total nitrogen content by the raw complete food allergen substance supplier) with the observed concentration. TABLE 2 shows the extraction efficiencies of 15 raw complete food allergen substances.

TABLE 2
Extraction Efficiency of 15 Raw
Complete Food Allergen Substances

| Food | | | Avg. | Extraction |
|---|---|---|---|---|
| Allergen | | Expected | Observed | Efficiency |
| Substance | % Protein* | (mg/mL)** | (mg/mL) | (%) |
| Almond | 51.6 | 10.3 | 8.6 | 82.8 |
| Cashew | 35.5 | 7.1 | 7.0 | 99.2 |
| Cod* | 897 | 17.4 | 2.1 | 12.0 |
| Egg | 47.0 | 9.4 | 9.8 | 104.0 |
| Hazelnut | 36.1 | 7.2 | 6.6 | 91.4 |
| Milk | 85.1 | 17.0 | 12.5 | 73.3 |
| Peanut | 47.1 | 9.4 | 7.4 | 78.6 |
| Pecan | 35.1 | 7.0 | 4.5 | 63.5 |
| Pistachio | 41.1 | 8.2 | 4.6 | 56.2 |
| Salmon* | 48.0 | 9.6 | 1.1 | 11.7 |
| Sesame | 58 | 11.6 | 5.7 | 49.3 |
| Shrimp | 62 | 12.4 | 1.1 | 8.9 |
| Soy | 91.5 | 18.3 | 16.1 | 88.0 |
| Walnut | 43.5 | 8.7 | 6.2 | 70.8 |
| Wheat | 75.8 | 15.2 | 3.5 | 23.1 |

*as determined by total nitrogen content value provided by each raw complete food allergen substance supplier.
**based on the standard extraction of 50 mg of raw complete food allergen substance in 2.5 mL extraction buffer if 100% of the protein is extractable.

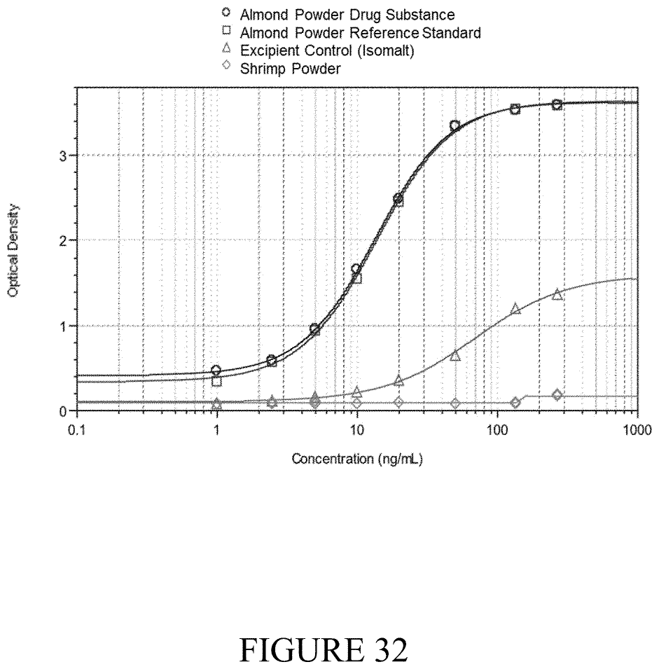

The amount of allergenic protein in each individual raw complete food allergen that is reactive with allergen-specific antibodies was measured by ELISA. As shown in FIG. 32, an almond powder drug substance (open circles) had similar reactivity in an almond protein-specific ELISA as compared to an almond powder reference standard (open squares). Non-specific food allergen substance (shrimp powder, open diamonds), and excipient control (isomalt, open triangles) were not reactive in the ELISA, demonstrating the specificity of the ELISA for the specific raw complete food allergen substance (i.e. almond). TABLE 3 shows EC₅₀ values of 15 food allergen substances determined using food allergen-specific ELISAs.

TABLE 3
EC₅₀ Values of 15 Raw Complete Food Allergen Substances

| Food | | |
|---|---|---|
| Allergen | EC₅₀ | Cross- |
| Substance | (ng/mL) | Reactivity |
| Almond | 18.7 | N/A |
| Cashew | 27.4 | Pistachio |
| Cod* | 660 | Salmon |
| Egg | 87.9 | N/A |
| Hazelnut | 136 | N/A |
| Milk | 219 | N/A |
| Peanut | 36.7 | N/A |
| Pecan | 275 | Walnut |
| Pistachio | 52.7 | Cashew |
| Salmon* | 1960 | Cod |
| Sesame | 38.4 | N/A |
| Shrimp | 130 | N/A |
| Soy | 188 | N/A |
| Walnut | 118 | Pecan |
| Wheat | 33.3 | N/A |

*Cod and salmon were both tested using the same commercially available ELISA

The protein profile of each individual raw complete food allergen was analyzed by comparing band profiles of each individual raw complete food allergen with corresponding raw complete food allergen standards by SDS-PAGE (described in Example 2).

### Example 2

The protein integrity of 15 allergen drug substances was compared to 15 corresponding raw complete food allergen substances. Fonterra bovine milk protein isolate 4900 and Michael Foods hen's egg powder were each separately irradiated with two doses of beta radiation at 7.5 kGy and 15 kGy (E-Beam irradiation at SADEX Corporation, Sioux City, Iowa). Hazelnut powder, cashew powder, pistachio powder, walnut powder, pecan powder, almond powder, peanut powder, sesame powder, soy protein powder, wheat protein powder, salmon powder, cod powder, and shrimp powder were each separately irradiated with 7.5 kGy of beta radiation (E-Beam irradiation at Steri-Tek, Fremont, California). Protein integrity was assessed by resolving all proteins present in both raw complete food allergen substance samples and irradiated allergen drug substance samples by SDS-PAGE. In brief, defined amounts of each of the 15 different raw complete food allergen substances and individual allergen drug substances were solubilized using lithium dodecyl sulfate and briefly heated for several minutes followed by centrifugation. Supernatants were then subjected to electrophoresis on 4-12% Bis-Tris Polyacrylamide gels under reducing conditions followed by staining with Coomassie Blue.

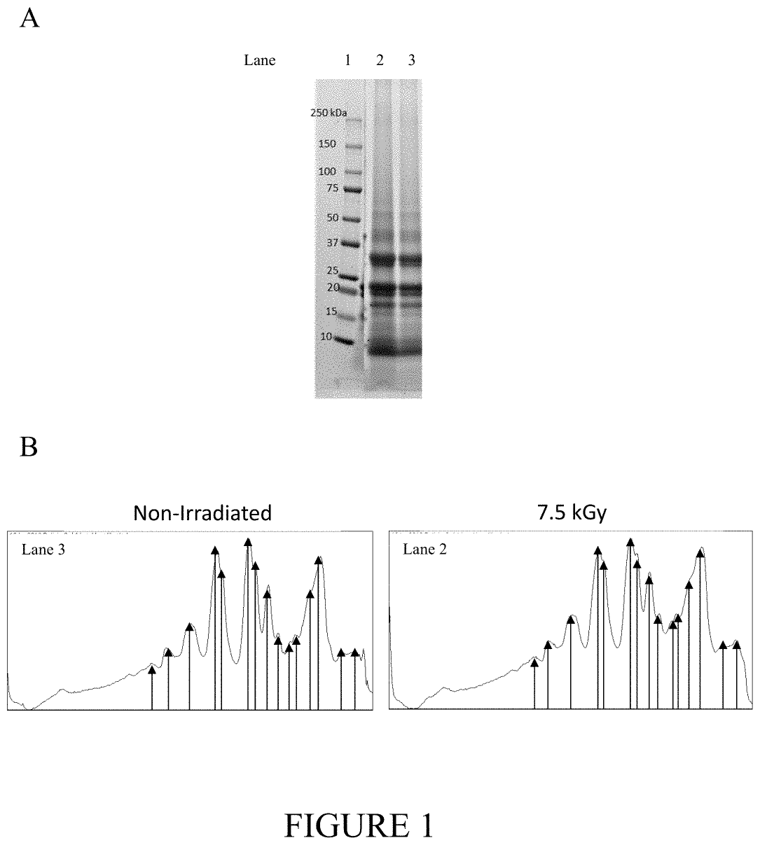

FIG. 1A shows that non-irradiated raw complete hazelnut allergen substance sample (lane 3) had very good banding resolution by SDS-PAGE and exhibited a similar protein band configuration as 7.5 kGy beta-irradiated hazelnut allergen drug substance sample (lane 2).

FIG. 1B shows the optical densitometry analysis of the SDS-PAGE of FIG. 1A. 15 protein band peaks were quantified in both 7.5 kGy beta-irradiated hazelnut allergen drug substance sample (lane 2 of FIG. 1A) and the non-irradiated raw complete hazelnut allergen substance sample (lane 3 of FIG. 1A), suggesting that protein integrity is highly conserved following beta radiation treatment at 7.5 kGy.

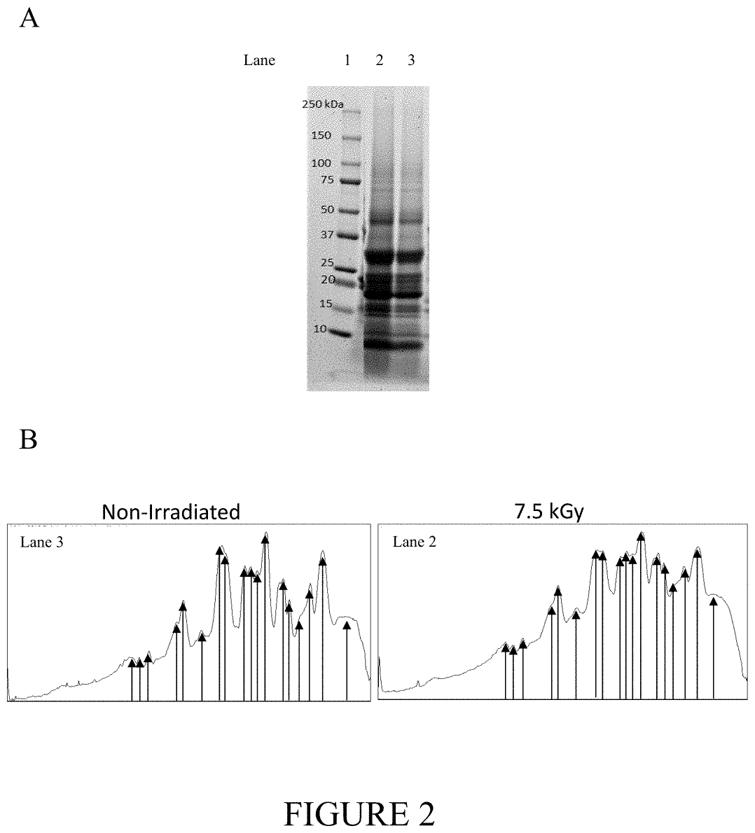

FIG. 2A shows that non-irradiated raw complete cashew allergen substance sample (lane 3) had very good banding resolution by SDS-PAGE and exhibited a similar protein band configuration as 7.5 kGy beta-irradiated cashew allergen drug substance sample (lane 2).

FIG. 2B shows the optical densitometry analysis of the SDS-PAGE of FIG. 2A. 18 protein band peaks were quantified in both 7.5 kGy beta-irradiated cashew allergen drug substance sample (lane 2 of FIG. 2A) and the non-irradiated raw complete cashew allergen substance sample (lane 3 of FIG. 2A), suggesting that protein integrity is highly conserved following beta radiation treatment at 7.5 kGy.

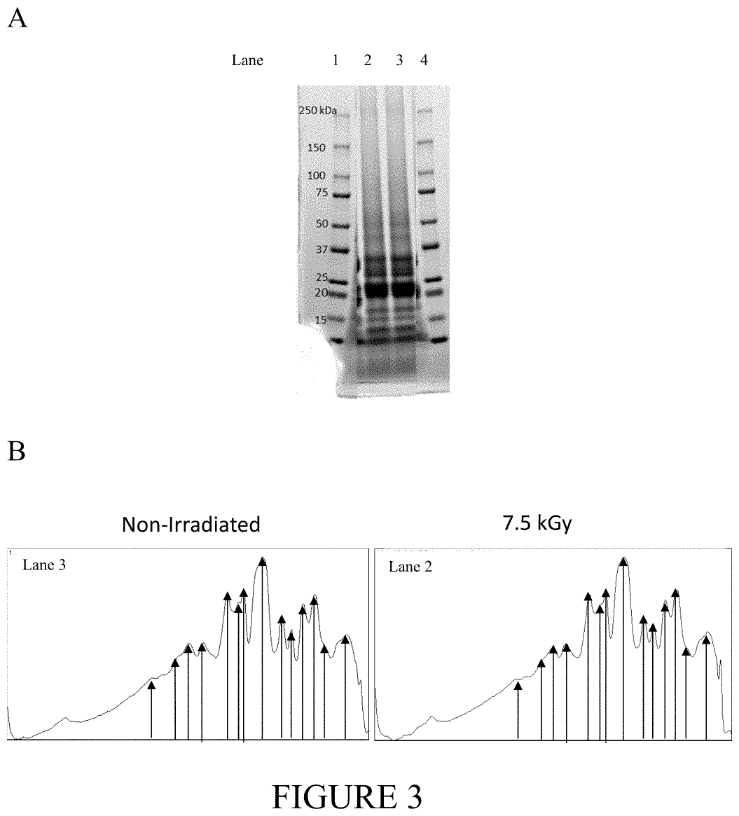

FIG. 3A shows that non-irradiated raw complete pistachio allergen substance sample (lane 3) had very good banding resolution by SDS-PAGE and exhibited a similar protein band configuration as 7.5 kGy beta-irradiated pistachio allergen drug substance sample (lane 2).

FIG. 3B shows the optical densitometry analysis of the SDS-PAGE of FIG. 3A. 14 protein band peaks were quantified in both the 7.5 kGy beta-irradiated pistachio allergen drug substance sample (lane 2 of FIG. 3A) and the non-irradiated raw complete pistachio allergen substance sample (lane 3 of FIG. 3A), suggesting that protein integrity is highly conserved following beta radiation treatment at 7.5 kGy.

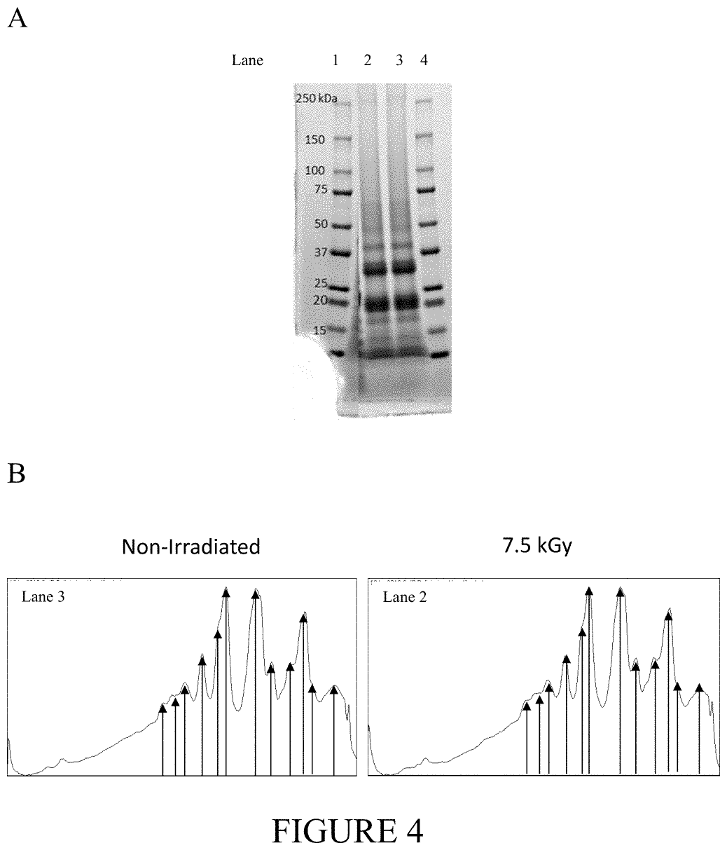

FIG. 4A shows that non-irradiated raw complete walnut allergen substance sample (lane 3) had very good banding resolution by SDS-PAGE and exhibited a similar protein band configuration as 7.5 kGy beta-irradiated cashew allergen drug substance sample (lane 2).

FIG. 4B shows the optical densitometry analysis of the SDS-PAGE of FIG. 4A. 12 protein band peaks were quantified in both the 7.5 kGy beta-irradiated walnut allergen drug substance sample (lane 2 of FIG. 4A) and the non-irradiated raw complete walnut allergen substance sample (lane 3 of FIG. 4A), suggesting that protein integrity is highly conserved following beta radiation treatment at 7.5 kGy.

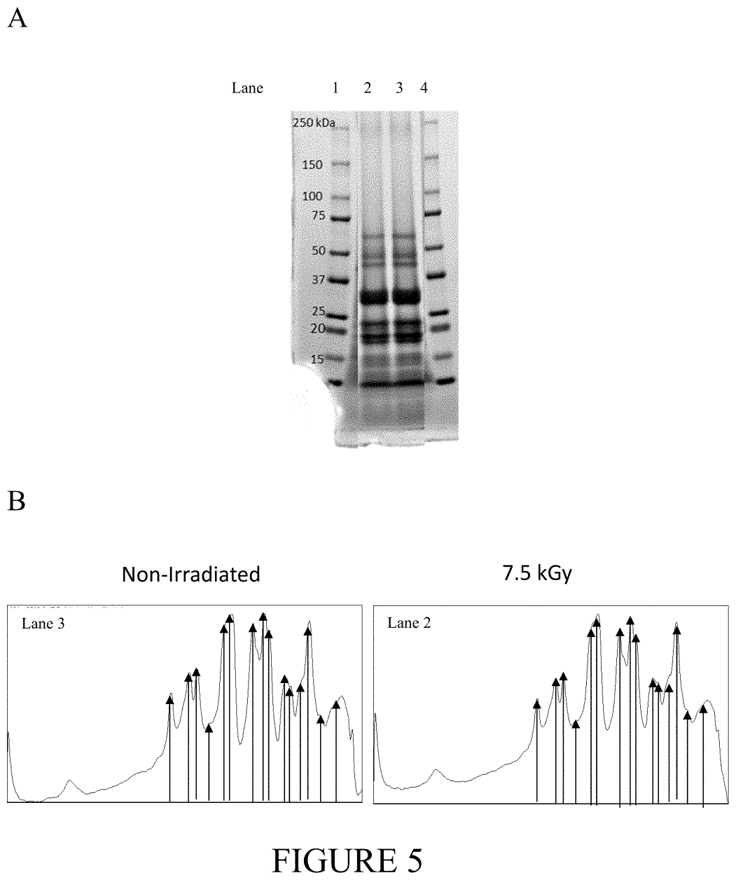

FIG. 5A shows that non-irradiated raw complete pecan allergen substance sample (lane 3) had very good banding resolution by SDS-PAGE and exhibited a similar protein band configuration as 7.5 kGy beta-irradiated pecan allergen drug substance sample (lane 2).

FIG. 5B shows the optical densitometry analysis of the SDS-PAGE of FIG. 5A. 15 protein band peaks were quantified in both the 7.5 kGy beta-irradiated pecan allergen drug substance sample (lane 2, FIG. 5A) and the non-irradiated raw complete pecan allergen substance sample (lane 3 of FIG. 5A), suggesting that protein integrity is highly conserved following beta radiation treatment at 7.5 kGy.

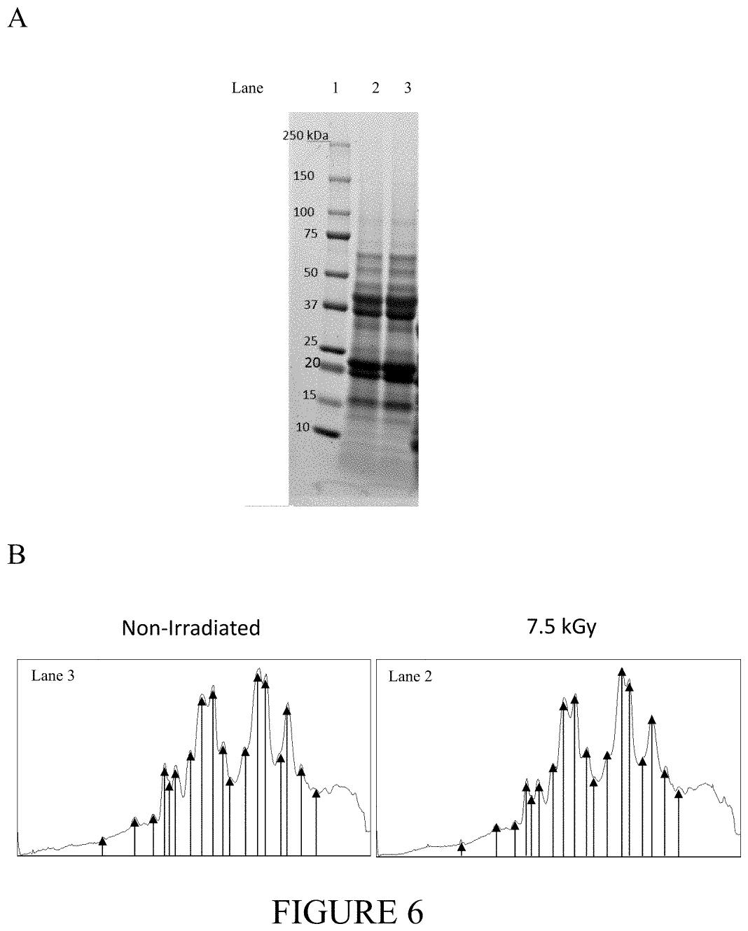

FIG. 6A shows that non-irradiated raw complete almond allergen substance sample (lane 3) had very good banding resolution by SDS-PAGE and exhibited a similar protein band configuration as 7.5 kGy beta-irradiated almond allergen drug substance sample (lane 2).

FIG. 6B shows the optical densitometry analysis of the SDS-PAGE of FIG. 6A. 18 protein band peaks were quantified in both the 7.5 kGy beta-irradiated almond allergen drug substance sample (lane 2 of FIG. 6A) and the non-irradiated raw complete almond allergen substance sample (lane 3 of FIG. 6A), suggesting that protein integrity is highly conserved following beta radiation treatment at 7.5 kGy.

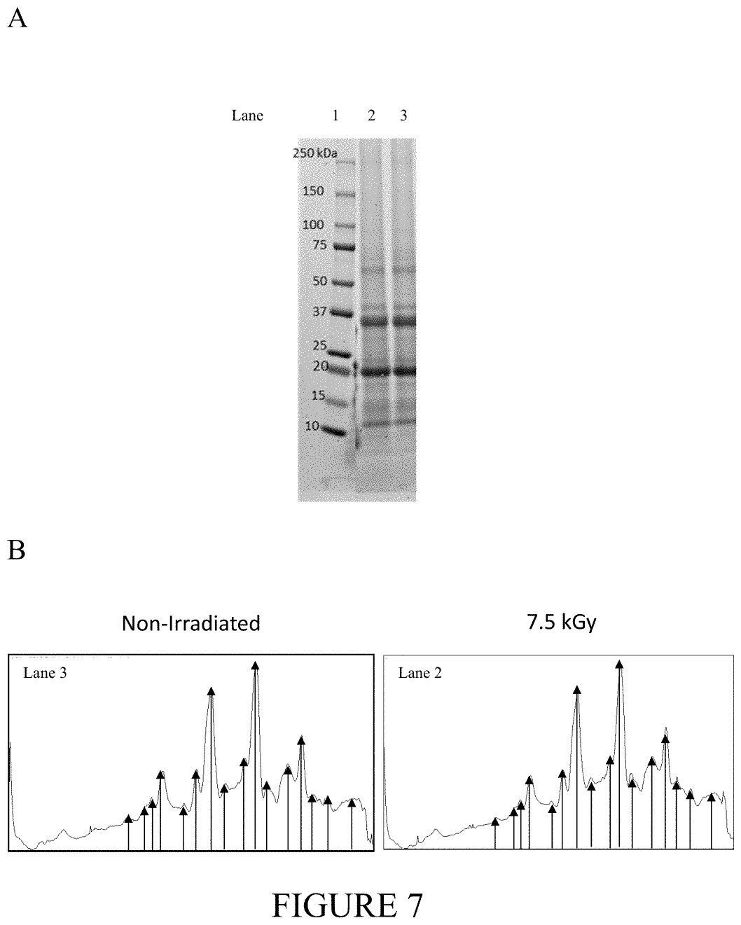

FIG. 7A shows that non-irradiated raw complete peanut allergen substance sample (lane 3) had good banding resolution by SDS-PAGE and exhibited a similar protein band configuration as 7.5 kGy beta-irradiated peanut allergen drug substance sample (lane 2).

FIG. 7B shows the optical densitometry analysis of the SDS-PAGE of FIG. 7A. 16 protein band peaks were quantified in both the 7.5 kGy beta-irradiated peanut allergen drug substance (lane 2 of FIG. 7A) and the non-irradiated raw complete peanut allergen substance sample (lane 3 of FIG. 7A), suggesting that protein integrity is highly conserved following beta radiation treatment at 7.5 kGy.

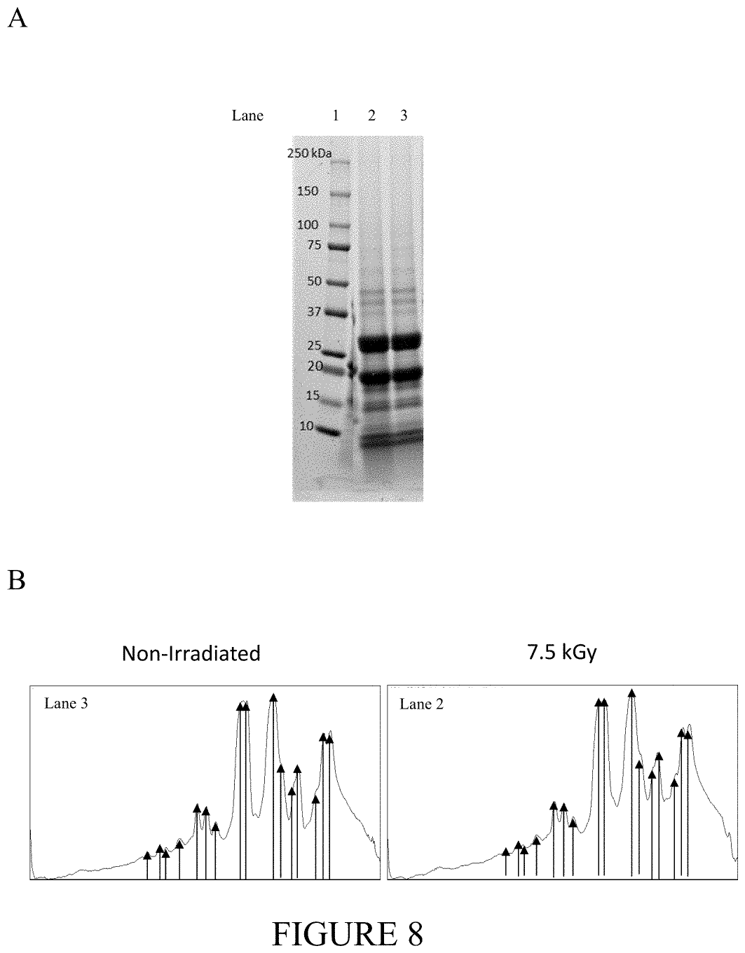

FIG. 8A shows that non-irradiated raw complete sesame allergen substance sample (lane 3) had very good banding resolution by SDS-PAGE and exhibited a similar protein band configuration as 7.5 kGy beta-irradiated sesame allergen drug substance sample (lane 2).

FIG. 8B shows the optical densitometry analysis of the SDS-PAGE of FIG. 8A. 16 protein band peaks were quantified in both the 7.5 kGy beta-irradiated sesame allergen drug substance sample (lane 2 of FIG. 8A) and the non-irradiated raw complete sesame substance allergen sample (lane 3 of FIG. 8A), suggesting that protein integrity is highly conserved following beta radiation treatment at 7.5 kGy.

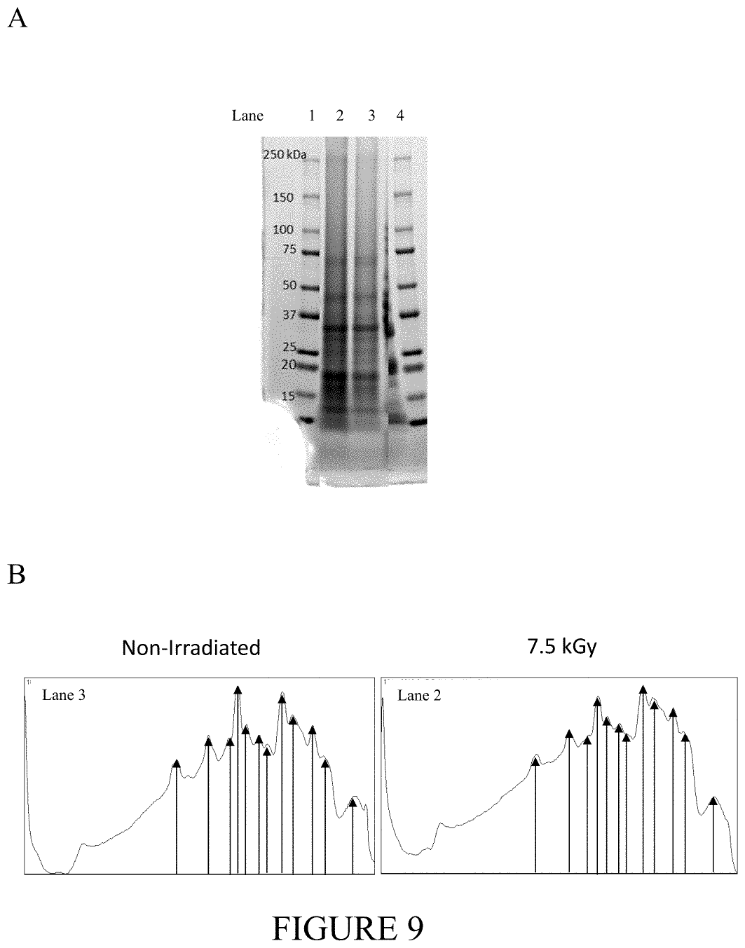

FIG. 9A shows that non-irradiated raw complete soy allergen substance sample (lane 3) had relatively low banding resolution by SDS-PAGE and exhibited a similar protein band configuration as 7.5 kGy beta-irradiated soy allergen drug substance sample (lane 2).

FIG. 9B shows the optical densitometry analysis of the SDS-PAGE of FIG. 9A. 12 protein band peaks were quantified in both the 7.5 kGy beta-irradiated soy allergen drug substance sample (lane 2 of FIG. 9A) and the non-irradiated raw complete soy allergen substance sample (lane 3 of FIG. 9A), suggesting that protein integrity is highly conserved following beta radiation treatment at 7.5 kGy.

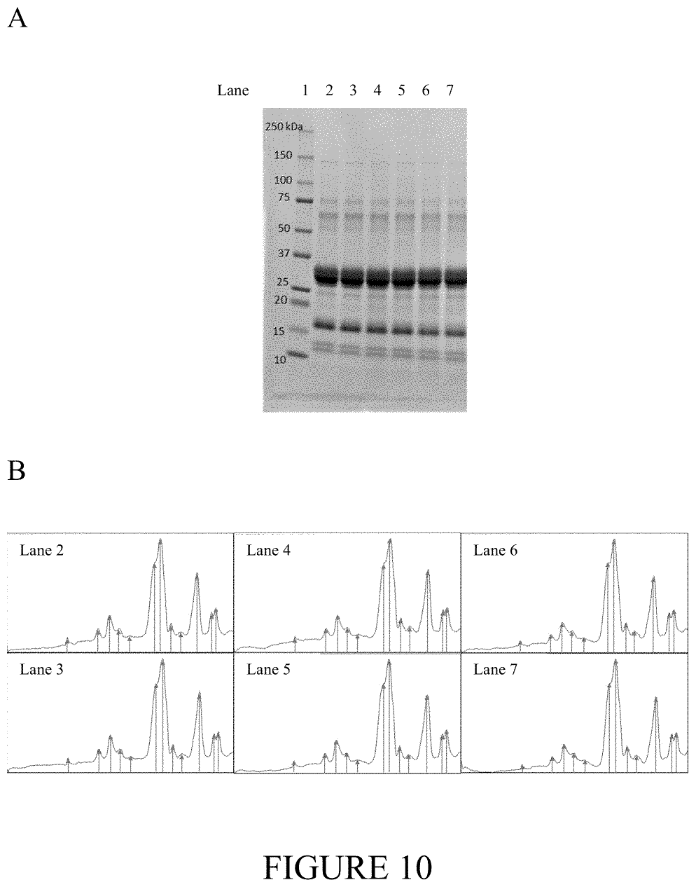

FIG. 10A shows that non-irradiated raw complete hen's egg allergen substance samples (lanes 2 and 3) had good banding resolution by SDS-PAGE and exhibited a similar protein band configuration as 7.5 kGy beta-irradiated hen's egg allergen drug substance samples (lanes 4 and 5) and 15 kGy beta-irradiated hen's egg allergen drug substance samples (lanes 6 and 7).

FIG. 10B shows the optical densitometry analysis of the SDS-PAGE of FIG. 10A. 16 protein band peaks were quantified in the non-irradiated raw complete hen's egg allergen samples (lanes 2 and 3 of FIG. 10A). The 7.5 kGy beta-irradiated hen's egg allergen drug substance samples (lanes 4 and 5 of FIG. 2A) and the 15 kGy beta-irradiated hen's egg allergen drug substance samples (lanes 6 and 7 of FIG. 2A) exhibited very similar peak intensity and positioning, suggesting that protein integrity is highly conserved following beta radiation treatment at 7.5 kGy and 15 kGy.

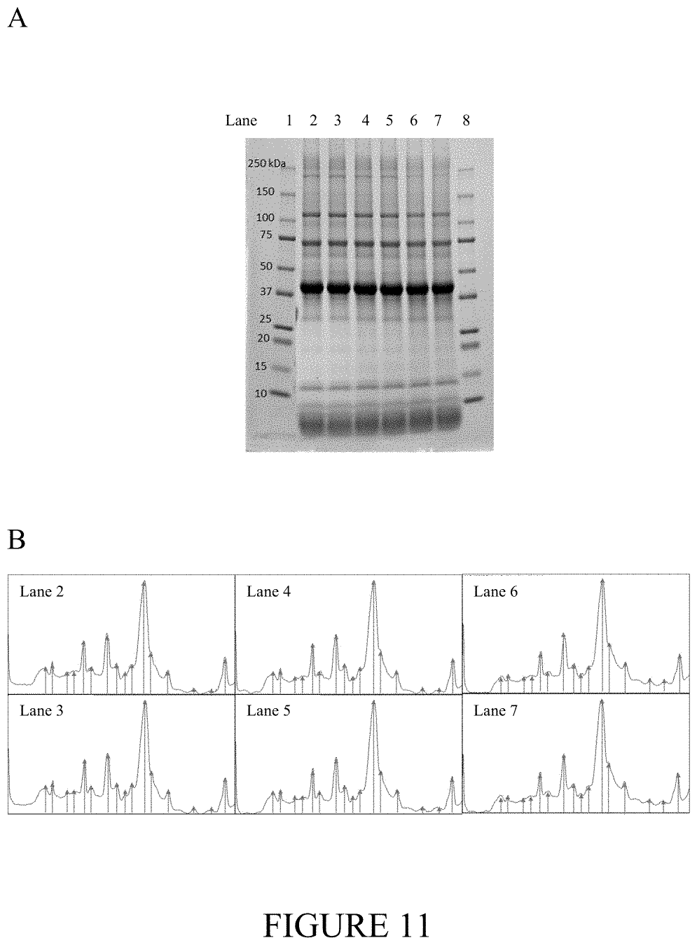

As shown in FIG. 11A, non-irradiated raw complete bovine milk allergen substance samples (lanes 2 and 3) had good banding resolution by SDS-PAGE and exhibited a similar protein band configuration as 7.5 kGy beta-irradiated bovine milk allergen drug substance samples (lanes 4 and 5), and 15 kGy beta-irradiated bovine milk allergen drug substance samples (lanes 6 and 7).

FIG. 11B shows optical densitometry analysis of the SDS-PAGE of FIG. 11A. 12 protein band peaks were quantified in the non-irradiated raw complete bovine milk allergen substance samples (lanes 2 and 3 of FIG. 11A). The 7.5 kGy beta-irradiated bovine milk allergen drug substance samples (lanes 4 and 5 of FIG. 11A) and the 15 kGy beta-irradiated bovine milk allergen drug substance samples (lanes 6 and 7 of FIG. 11A) exhibited very similar peak intensity and positioning, suggesting that protein integrity is highly conserved following beta radiation treatment at 7.5 kGy and 15 kGy.

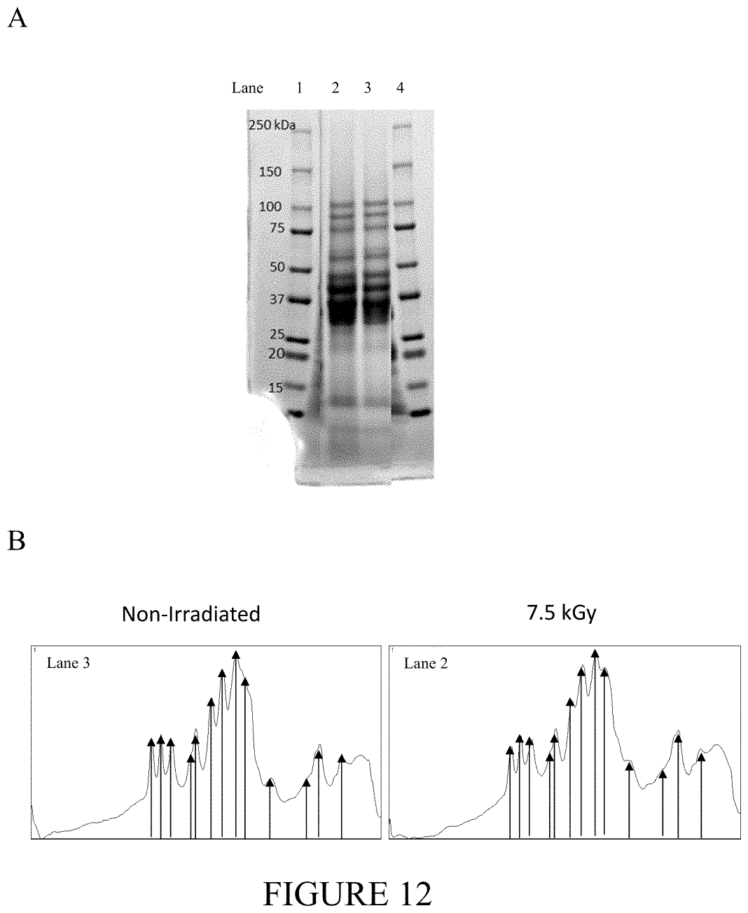

FIG. 12A shows that non-irradiated raw complete wheat allergen substance sample (lane 3) had very good banding resolution by SDS-PAGE and exhibited a similar protein band configuration as 7.5 kGy beta-irradiated wheat allergen drug substance sample (lane 2).

FIG. 12B shows the optical densitometry analysis of the SDS-PAGE of FIG. 12A. 13 protein band peaks were quantified in both the 7.5 kGy beta-irradiated wheat allergen drug substance sample (lane 2 of FIG. 12A) and the non-irradiated raw complete wheat allergen substance sample (lane 3 of FIG. 12A), suggesting that protein integrity is highly conserved following beta radiation treatment at 7.5 kGy.

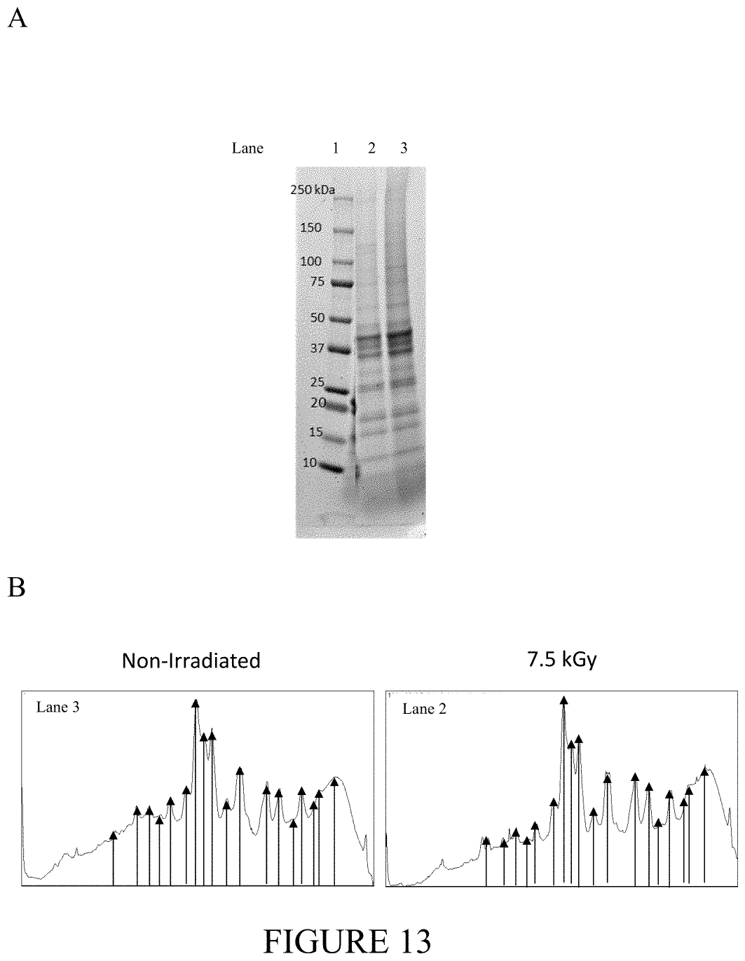

FIG. 13A shows that non-irradiated raw complete salmon allergen substance sample (lane 3) had very good banding resolution by SDS-PAGE and exhibited a similar protein band configuration as 7.5 kGy beta-irradiated salmon allergen drug substance sample (lane 2).

FIG. 13B shows the optical densitometry analysis of the SDS-PAGE of FIG. 13A. 18 protein band peaks were quantified in both the 7.5 kGy beta-irradiated salmon allergen drug substance sample (lane 2 of FIG. 13A) and the non-irradiated raw complete salmon allergen substance sample (lane 3 of FIG. 13A), suggesting that protein integrity is highly conserved following beta radiation treatment at 7.5 kGy.

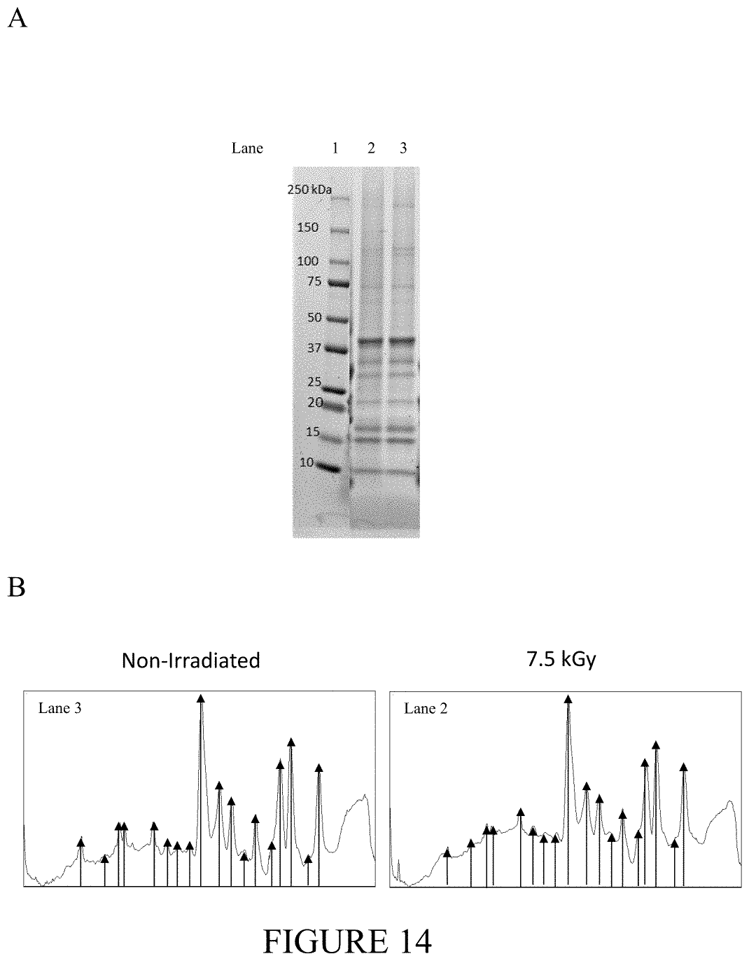

FIG. 14A shows that non-irradiated raw complete cod allergen substance sample (lane 3) had good banding resolution by SDS-PAGE and exhibited a similar protein band configuration as 7.5 kGy beta-irradiated cod allergen drug substance sample (lane 2).

FIG. 14B shows the optical densitometry analysis of the SDS-PAGE of FIG. 14A. 18 protein band peaks were quantified in both the 7.5 kGy beta-irradiated cod allergen drug substance sample (lane 2 of FIG. 14A) and the non-irradiated raw complete cod allergen substance sample (lane 3 of FIG. 14A), suggesting that protein integrity is highly conserved following beta radiation treatment at 7.5 kGy.

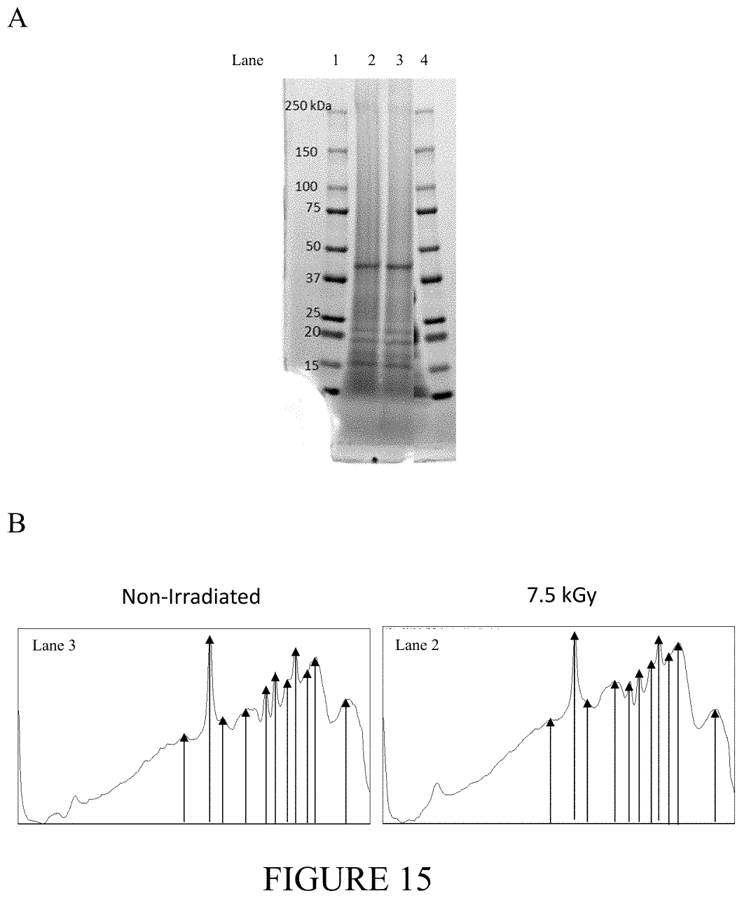

FIG. 15A shows that non-irradiated raw complete shrimp allergen substance sample (lane 3) had relatively low banding resolution by SDS-PAGE and exhibited a similar protein band configuration as 7.5 kGy beta-irradiated shrimp allergen drug substance sample (lane 2).

FIG. 15B shows the optical densitometry analysis of the SDS-PAGE of FIG. 15A. 11 protein band peaks were quantified in both the 7.5 kGy beta-irradiated shrimp allergen drug substance sample (lane 2 of FIG. 15A) and the non-irradiated raw shrimp allergen samples (lane 3 of FIG. 15A), suggesting that protein integrity is highly conserved following beta radiation treatment at 7.5 kGy.

### Example 3

The particle size distribution of 15 representative allergen drug substances was compared to 15 corresponding raw complete food allergen substances.

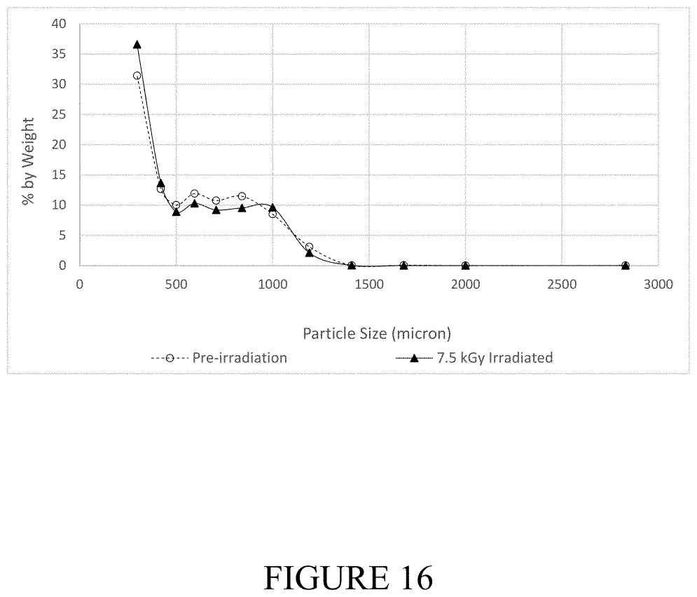

As shown in FIG. 16, treatment of raw complete hazelnut allergen substance with 7.5 kGy (closed triangles) of beta radiation did not result in significant differences in particle size distribution as compared to non-irradiated raw complete hazelnut allergen substance (open circles).

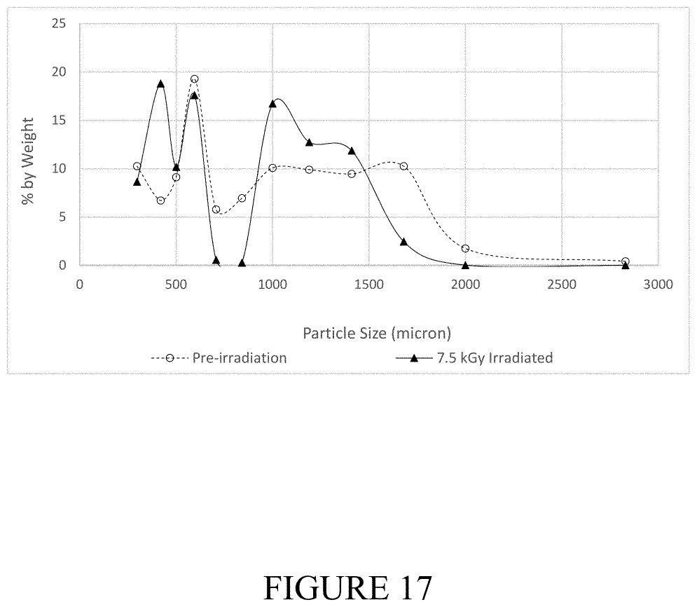

As shown in FIG. 17, treatment of raw complete cashew allergen substance with 7.5 kGy (closed triangles) of beta radiation did not result in significant differences in particle size distribution as compared to non-irradiated raw complete cashew allergen substance (open circles).

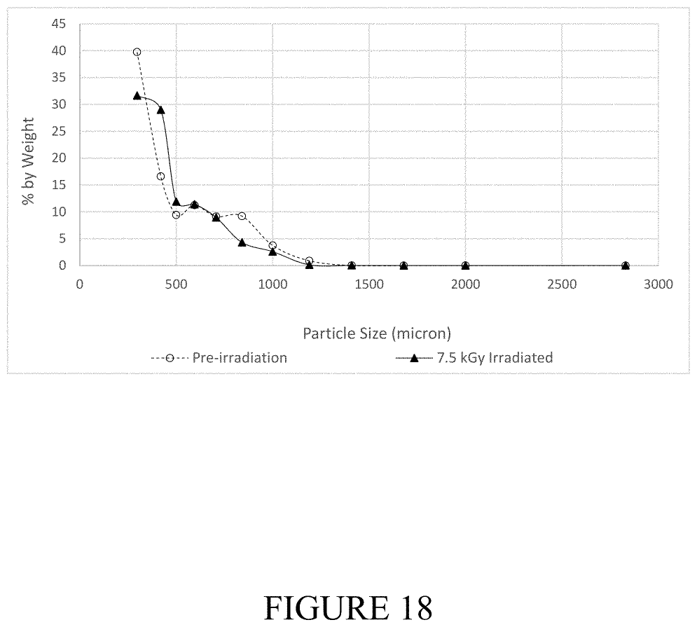

As shown in FIG. 18, treatment of raw complete pistachio allergen substance with 7.5 kGy (closed triangles) of beta radiation did not result in significant differences in particle size distribution as compared to non-irradiated raw complete pistachio allergen substance (open circles).

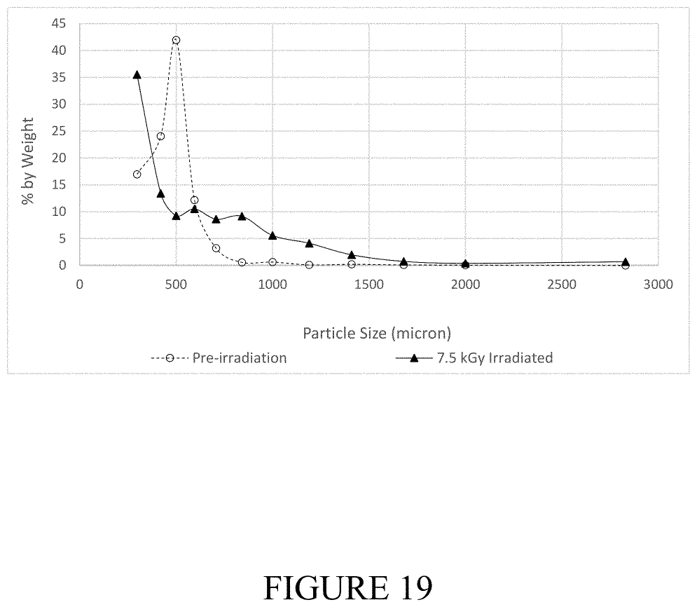

Treatment of raw complete walnut allergen substance with 7.5 kGy (FIG. 19; closed triangles) of beta radiation did not result in significant differences in particle size distribution as compared to non-irradiated raw complete walnut allergen substance (FIG. 19; open circles).

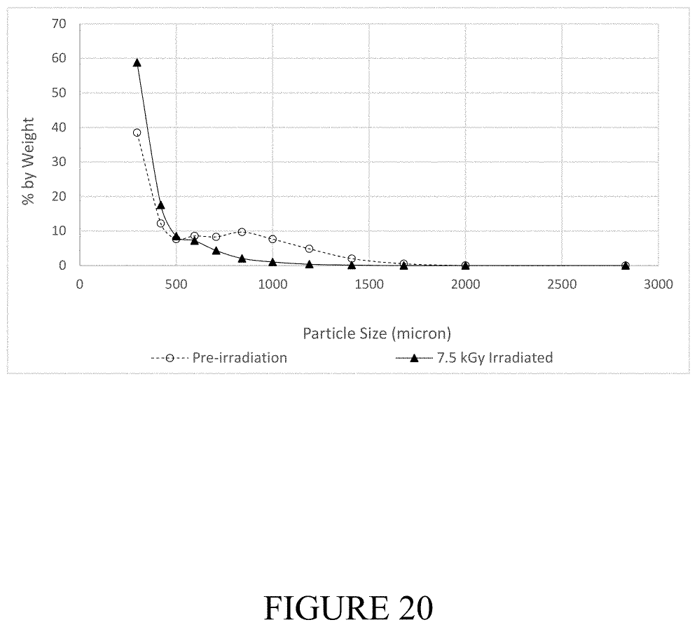

As shown in FIG. 20, treatment of raw complete pecan allergen substance with 7.5 kGy (closed triangles) of beta radiation did not result in significant differences in particle size distribution as compared to non-irradiated raw complete pecan allergen substance (open circles).

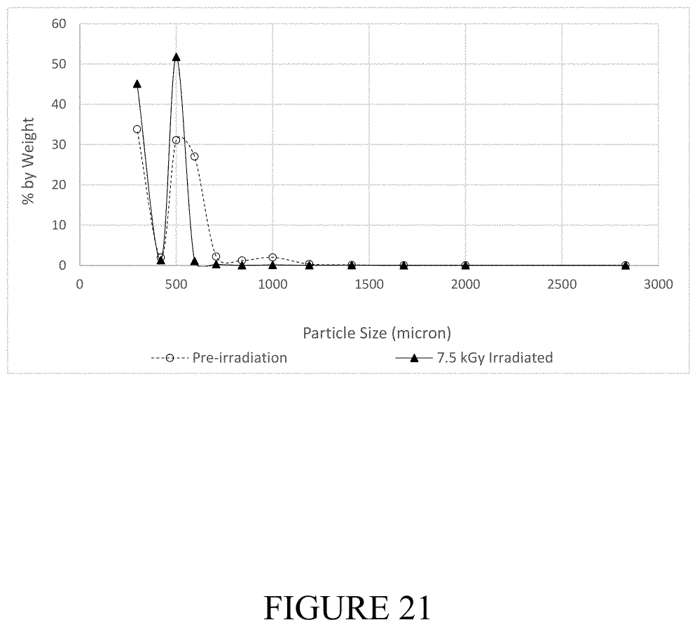

As shown in FIG. 21, treatment of raw complete almond allergen substance with 7.5 kGy (closed triangles) of beta radiation did not result in significant differences in particle size distribution as compared to non-irradiated raw complete almond allergen substance (open circles).

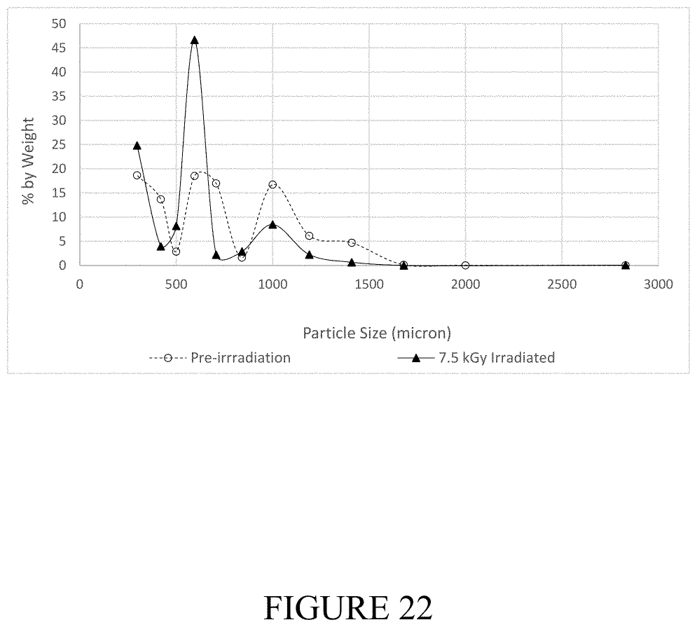

As shown in FIG. 22, treatment of raw complete peanut allergen substance with 7.5 kGy (closed triangles) of beta radiation did not result in significant differences in particle size distribution as compared to non-irradiated raw complete peanut allergen substance (open circles). There is a higher population of particles between 500 μm and 750 μm measured post-irradiation.

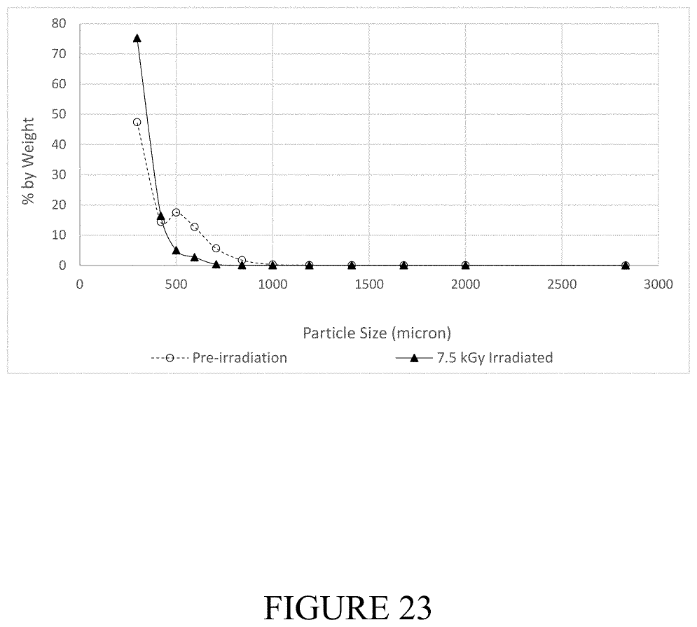

As shown in FIG. 23, treatment of raw complete sesame allergen substance with 7.5 kGy (closed triangles) of beta radiation did not result in significant differences in particle size distribution as compared to non-irradiated raw complete sesame allergen substance (open circles).

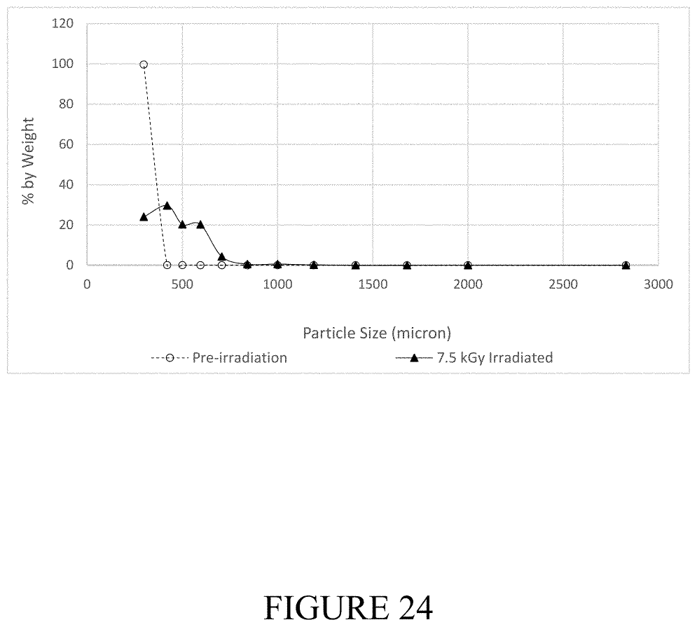

As shown in FIG. 24, treatment of raw complete soy allergen substance with 7.5 kGy (closed triangles) of beta radiation did not result in significant differences in particle size distribution as compared to non-irradiated raw complete soy allergen substance (open circles).

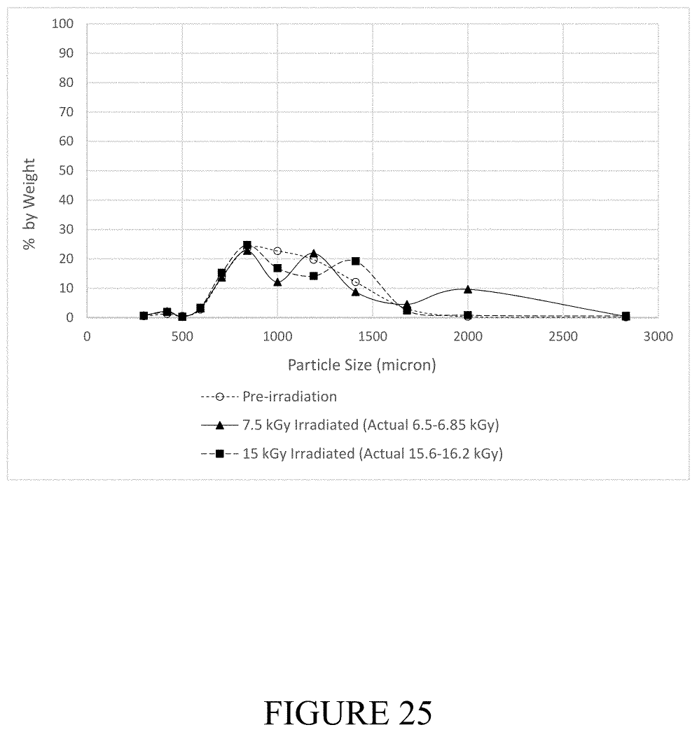

Similarly, as shown in FIG. 25, non-irradiated raw complete hen's egg allergen substance (open circles), 7.5 kGy beta-irradiated hen's egg allergen drug substance (closed triangles), and 15 kGy beta-irradiated hen's egg allergen drug substance (closed squares), did not exhibit significant differences in particle size distribution.

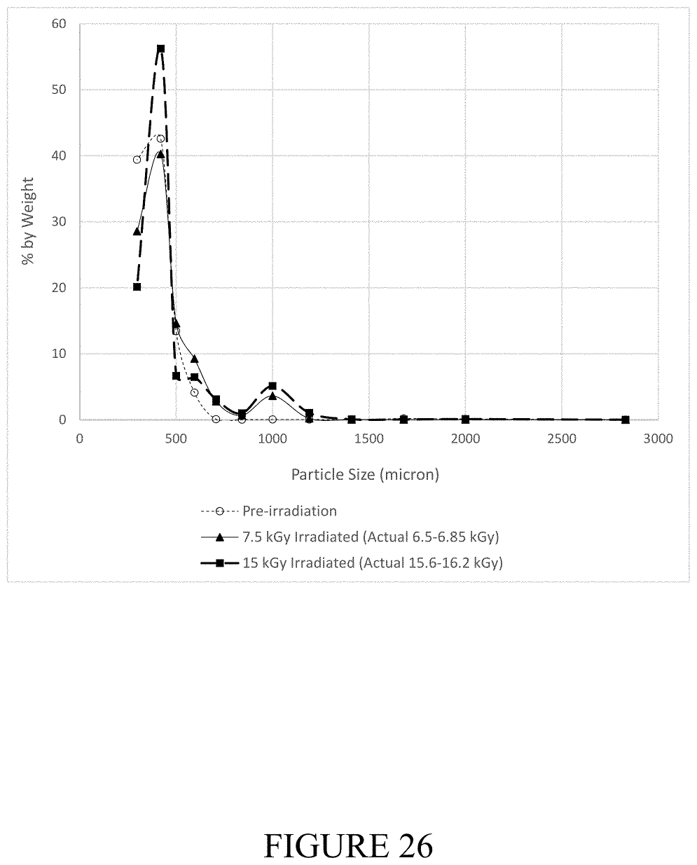

As shown in FIG. 26, treatment of raw complete bovine milk allergen substance with 7.5 kGy (closed triangles) or 15 kGy (closed squares) of beta radiation did not result in significant differences in particle size distribution as compared to non-irradiated raw complete bovine milk allergen substance (open circles).

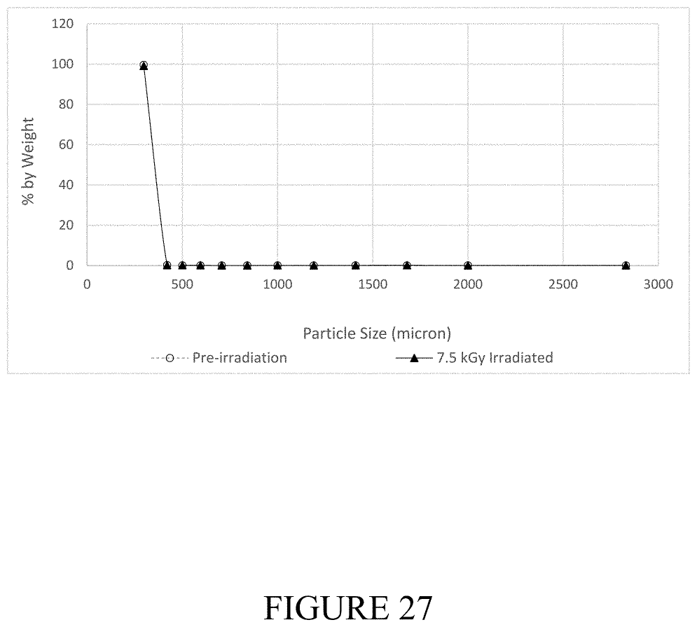

As shown in FIG. 27, treatment of raw complete wheat allergen substance with 7.5 kGy (closed triangles) of beta radiation did not result in significant differences in particle size distribution as compared to non-irradiated raw complete wheat allergen substance (open circles).

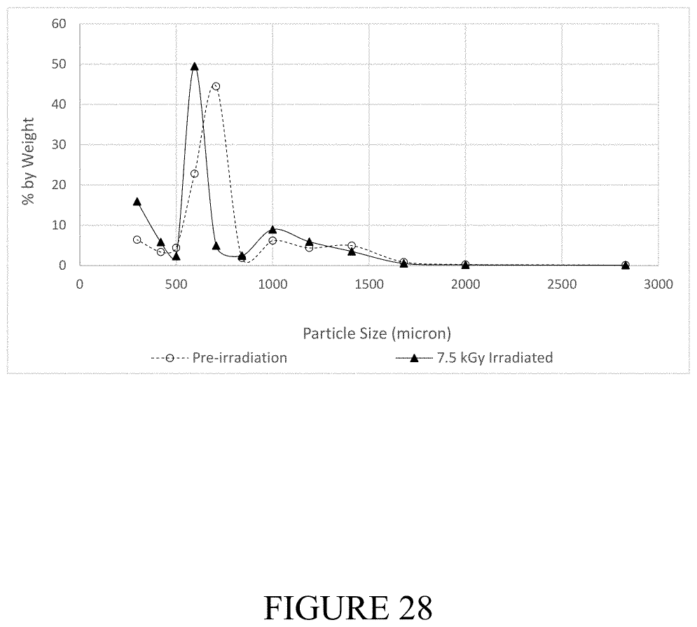

As shown in FIG. 28, treatment of raw complete salmon allergen substance with 7.5 kGy (closed triangles) of beta radiation did not result in significant differences in particle size distribution as compared to non-irradiated raw complete salmon allergen substance (open circles).

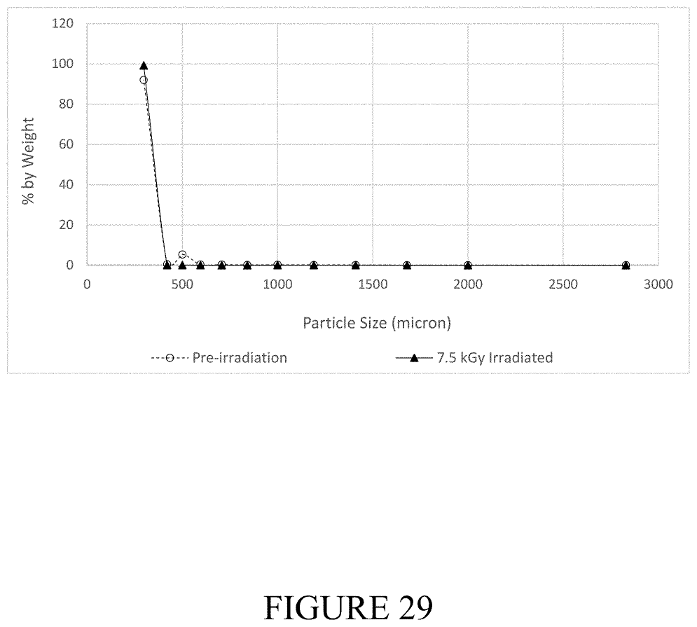

Treatment of raw complete cod allergen substance with 7.5 kGy (FIG. 29; closed triangles) of beta radiation did not result in significant differences in particle size distribution as compared to non-irradiated raw complete cod allergen substance (FIG. 29; open circles).

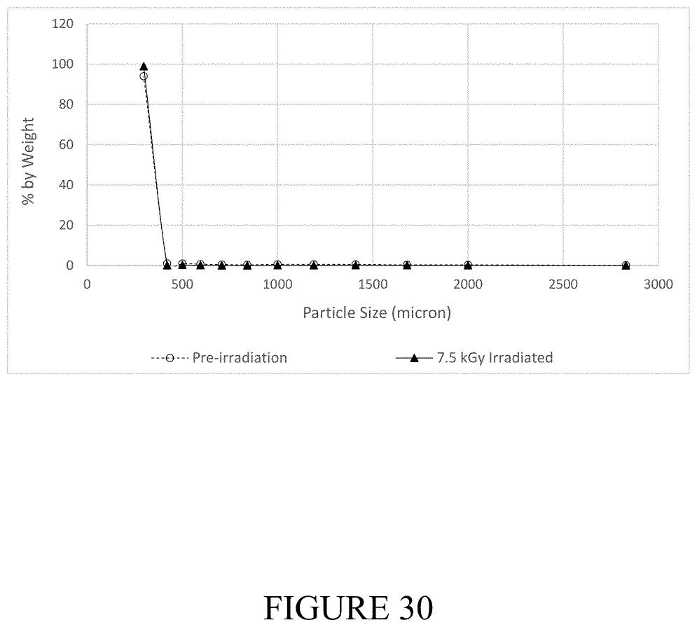

As shown in FIG. 30, treatment of raw complete shrimp allergen substance with 7.5 kGy (closed triangles) of beta radiation did not result in significant differences in particle size distribution as compared to non-irradiated raw complete shrimp allergen substance (open circles).

Thus, systematic agglomeration does not appear to occur in irradiated allergen drug substances, even after high (e.g., 15 kGy for some allergen substances) doses of beta radiation.

### Example 4

Moisture content and water activity were also measured for 15 representative allergen drug substances and compared to the moisture content and water activity of 15 corresponding raw complete food allergen substances.

As shown in TABLE 4, moisture content (as measured using AquaLab 4TE and Computract 1000XL) and water activity (as measured using Rotronic HygroLab) of raw complete hazelnut allergen substance were similar pre-beta radiation treatment and post-beta radiation treatment.

TABLE 4

| Hazelnut | | |
|---|---|---|
| Allergen | Non- | 7.5 kGy Beta |
| Substance | irradiated | Radiation Dose |
| Moisture (%): | 4.03 | 3.77 |
| Water activity | 0.288 | 0.273 |

As shown in TABLE 5, moisture content (as measured using AquaLab 4TE and Computract 1000XL) and water activity (as measured using Rotronic HygroLab) of raw complete cashew allergen substance were similar pre-beta radiation treatment and post-beta radiation treatment.

TABLE 5

| Cashew | | |
|---|---|---|
| Allergen | Non- | 7.5 kGy Beta |
| Substance | irradiated | Radiation Dose |
| Moisture (%): | 5.91 | 6.18 |
| Water activity | 0.356 | 0.323 |

As shown in TABLE 6, moisture content (as measured using AquaLab 4TE and Computract 1000XL) and water activity (as measured using Rotronic HygroLab) of raw complete pistachio allergen substance were similar pre-beta radiation treatment and post-beta radiation treatment (by 8% for both).

TABLE 6

| Pisatachio | | |
|---|---|---|
| Allergen | Non- | 7.5 kGy Beta |
| Substance | irradiated | Radiation Dose |
| Moisture (%): | 2.83 | 2.59 |
| Water activity | 0.213 | 0.195 |

As shown in TABLE 7, moisture content (as measured using AquaLab 4TE and Computract 1000XL) and water activity (as measured using Rotronic HygroLab) of raw complete walnut allergen substance were not systematically affected by beta radiation treatment.

TABLE 7

| Walnut | | |
|---|---|---|
| Allergen | Non- | 7.5 kGy Beta |
| Substance | irradiated | Radiation Dose |
| Moisture (%): | 3.42 | 3.31 |
| Water activity | 0.239 | 0.243 |

As shown in TABLE 8, moisture content (as measured using AquaLab 4TE and Computract 1000XL) and water activity (as measured using Rotronic HygroLab) of raw complete pecan allergen substance were similar post-beta radiation treatment (by 5% and 4%, respectively).

TABLE 8

| Pecan | | |
|---|---|---|
| Allergen | Non- | 7.5 kGy Beta |
| Substance | irradiated | Radiation Dose |
| Moisture (%): | 5.85 | 6.15 |
| Water activity | 0.354 | 0.371 |

As shown in TABLE 9, moisture content (as measured using AquaLab 4TE and Computract 1000XL) and water activity (as measured using Rotronic HygroLab) of raw complete almond allergen substance were similar pre-beta radiation treatment and post-beta radiation treatment (by 5% and 11%, respectively).

TABLE 9

| Pecan | | |
|---|---|---|
| Allergen | Non- | 7.5 kGy Beta |
| Substance | irradiated | Radiation Dose |
| Moisture (%): | 6.39 | 6.74 |
| Water activity | 0.366 | 0.409 |

As shown in TABLE 10, moisture content (as measured using AquaLab 4TE and Computract 1000XL) and water activity (as measured using Rotronic HygroLab) of raw complete peanut allergen substance were similar pre-beta radiation treatment and post-beta radiation treatment.

TABLE 10

| Peanut | | |
|---|---|---|
| Allergen | Non- | 7.5 kGy Beta |
| Substance | irradiated | Radiation Dose |
| Moisture (%): | 2.02 | 2.08 |
| Water activity | 0.105 | 0.133 |

As shown in TABLE 11, moisture content (as measured using AquaLab 4TE and Computract 1000XL) and water activity (as measured using Rotronic HygroLab) of raw complete sesame allergen substance were similar pre-beta radiation treatment and post-beta radiation treatment.

TABLE 11

| Sesame | | |
|---|---|---|
| Allergen | Non- | 7.5 kGy Beta |
| Substance | irradiated | Radiation Dose |
| Moisture (%): | 3.33 | 3.41 |
| Water activity | 0.173 | 0.195 |

As shown in TABLE 12, moisture content (as measured using AquaLab 4TE and Computract 1000XL) and water activity (as measured using Rotronic HygroLab) of raw complete soy allergen substance were similar pre-beta radiation treatment and post-beta radiation treatment.

TABLE 12

| Soy | | |
|---|---|---|
| Allergen | Non- | 7.5 kGy Beta |
| Substance | irradiated | Radiation Dose |
| Moisture (%): | 6.12 | 6.66 |
| Water activity | 0.256 | 0.285 |

The results in TABLE 13, similarly show that moisture content and water activity of raw complete hen's egg allergen substance were not systematically affected by beta radiation treatment at either a 7.5 kGy or 15 kGy dose.

TABLE 13

| Hen's Egg | | | |
|---|---|---|---|
| Allergen | Non- | 7.5 kGy Beta | 15 kGy Beta |
| Substance | irradiated | Radiation Dose | Radiation Dose |
| Moisture (%): | 4.15 | 3.61 | 2.74 |
| Water activity | 0.426 | 0.364 | 0.373 |

As shown in TABLE 14, moisture content (as measured using AquaLab 4TE and Computract 1000XL) and water activity (as measured using Rotronic HygroLab) of raw complete bovine milk allergen substance were not systematically affected by beta radiation treatment at either a 7.5 kGy or 15 kGy dose.

TABLE 14

| Bovine Milk | | | |
|---|---|---|---|
| Allergen | Non- | 7.5 kGy Beta | 15 kGy Beta |
| Substance | irradiated | Radiation Dose | Radiation Dose |
| Moisture (%): | 4.88 | 5.83 | 5.09 |
| Water activity | 0.349 | 0.435 | 0.392 |

As shown in TABLE 15, moisture content (as measured using AquaLab 4TE and Computract 1000XL) and water activity (as measured using Rotronic HygroLab) of raw complete wheat allergen substance were slightly higher post-beta radiation treatment as compared to pre-beta radiation treatment.

TABLE 15

| Wheat | | |
|---|---|---|
| Allergen | Non- | 7.5 kGy Beta |
| Substance | irradiated | Radiation Dose |
| Moisture (%): | 5.8 | 6.68 |
| Water activity | 0.278 | 0.335 |

As shown in TABLE 16, moisture content (as measured using AquaLab 4TE and Computract 1000XL) and water activity (as measured using Rotronic HygroLab) of raw complete salmon allergen substance were similar pre-beta radiation treatment and post-beta radiation treatment.

TABLE 16

| Salmon | | |
|---|---|---|
| Allergen | Non- | 7.5 kGy Beta |
| Substance | irradiated | Radiation Dose |
| Moisture (%): | 3.32 | 3.28 |
| Water activity | 0.284 | 0.304 |

As shown in TABLE 17, moisture content (as measured using AquaLab 4TE and Computract 1000XL) and water activity (as measured using Rotronic HygroLab) of raw complete cod allergen substance were not systematically affected by beta radiation treatment at 7.5 kGy.

TABLE 17

| Cod | | |
|---|---|---|
| Allergen | Non- | 7.5 kGy Beta |
| Substance | irradiated | Radiation Dose |
| Moisture (%): | 4.79 | 4.89 |
| Water activity | 0.227 | 0.235 |

As shown in TABLE 18, moisture content (as measured using AquaLab 4TE and Computract 1000XL) and water activity (as measured using Rotronic HygroLab) of raw complete shrimp allergen substance were similar pre-beta radiation treatment and post-beta radiation treatment.

TABLE 18

| Shrimp | | |
|---|---|---|
| Allergen | Non- | 7.5 kGy Beta |
| Substance | irradiated | Radiation Dose |
| Moisture (%): | 3.6 | 4.97 |
| Water activity | 0.183 | 0.261 |

### Example 5

Microbial growth was measured for 15 representative allergen drug substances and compared to the microbial growth of 15 corresponding raw complete food allergen substances.

Total aerobic microorganisms were measured for non-irradiated and irradiated bovine milk allergen substance and hen's egg allergen substance samples. Additionally, samples were measured for total Enterobacteriaceae, yeast and mold counts.

For total aerobic plate counts and Enterobacteriaceae counts, samples were diluted and spread onto a petri dish of general recovery media to measure colony-forming units per gram of product (CFU/g). For yeast and mold plate counts, samples were diluted and spread onto 3M Petrifilm™ yeast and mold count plates in accordance with the manufacturer's instructions and colonies were reported as colony-forming units per gram of product (CFU/g).

The total aerobic organism plate counts, total Enterobacteriaceae counts, total yeast counts, and total mold counts for non-irradiated raw complete hazelnut allergen substance and 7.5 kGy beta-irradiated hazelnut allergen drug substance, are presented in TABLE 19. Irradiation with 7.5 kGy resulted in significant reduction (below the limit of detection) in the total aerobic and yeast counts.

TABLE 19

| | | |
|---|---|---|
| Hazelnut | | |
| Allergen | Non- | 7.5 kGy Beta |
| Substance | irradiated | Radiation Dose |

| | | | |
|---|---|---|---|
| Total Aerobic Plate | 1500 | CFU/g | <10 CFU/g |
| Count | | | |

| | | |
|---|---|---|
| Enterobacteriaceae | NA | <10 CFU/g |
| count | | |

| | | | |
|---|---|---|---|
| Yeast count | 160 | CFU/g | <10 CFU/g |
| Mold count | <10 | CFU/g | <10 CFU/g |

The total aerobic organism plate counts, total Enterobacteriaceae counts, total yeast counts, and total mold counts for non-irradiated raw complete cashew allergen substance and 7.5 kGy beta-irradiated cashew allergen drug substance, are presented in TABLE 20. Irradiation with 7.5 kGy resulted in significant reduction (below the limit of detection) in the total aerobic and mold counts.

TABLE 20

| | | |
|---|---|---|
| Cashew | | |
| Allergen | Non- | 7.5 kGy Beta |
| Substance | irradiated | Radiation Dose |

| | | | |
|---|---|---|---|
| Total Aerobic Plate | 13,000 | CFU/g | <10 CFU/g |
| Count | | | |
| Enterobacteriaceae | 20 | CFU/g | <10 CFU/g |
| count | | | |
| Yeast count | <10 | CFU/g | <10 CFU/g |
| Mold count | 360 | CFU/g | <10 CFU/g |

The total aerobic organism plate counts, total Enterobacteriaceae counts, total yeast counts, and total mold counts for non-irradiated raw complete pistachio allergen substance and 7.5 kGy beta-irradiated pistachio allergen drug substance, are presented in TABLE 21. Irradiation with 7.5 kGy resulted in significant reduction in the total aerobic plate count.

TABLE 21

| | | |
|---|---|---|
| Pistachio | | |
| Allergen | Non- | 7.5 kGy Beta |
| Substance | irradiated | Radiation Dose |

| | | | |
|---|---|---|---|
| Total Aerobic Plate | 760 | CFU/g | <10 CFU/g |
| Count | | | |

| | | |
|---|---|---|
| Enterobacteriaceae | NA | <10 CFU/g |
| count | | |

| | | | |
|---|---|---|---|
| Yeast count | <10 | CFU/g | <10 CFU/g |
| Mold count | <10 | CFU/g | <10 CFU/g |

The total aerobic organism plate counts, total Enterobacteriaceae counts, total yeast counts, and total mold counts for non-irradiated raw complete walnut allergen substance and 7.5 kGy beta-irradiated walnut allergen drug substance, are presented in TABLE 22. Irradiation with 7.5 kGy resulted in significant reduction in the total aerobic plate count.

TABLE 22

| | | |
|---|---|---|
| Walnut | | |
| Allergen | Non- | 7.5 kGy Beta |
| Substance | irradiated | Radiation Dose |

| | | | |
|---|---|---|---|
| Total Aerobic Plate | 140 | CFU/g | <10 CFU/g |
| Count | | | |

| | | |
|---|---|---|
| Enterobacteriaceae | NA | <10 CFU/g |
| count | | |

| | | | |
|---|---|---|---|
| Yeast count | <10 | CFU/g | <10 CFU/g |
| Mold count | <10 | CFU/g | <10 CFU/g |

The total aerobic organism plate counts, total Enterobacteriaceae counts, total yeast counts, and total mold counts for non-irradiated raw complete pecan allergen substance and 7.5 kGy beta-irradiated pecan allergen drug substance, are presented in TABLE 23. Irradiation with 7.5 kGy resulted in significant reduction in the total aerobic plate count, yeast count, and mold count.

TABLE 23

| | | |
|---|---|---|
| Pecan | | |
| Allergen | Non- | 7.5 kGy Beta |
| Substance | irradiated | Radiation Dose |

| | | | |
|---|---|---|---|
| Total Aerobic Plate | <100,000 | CFU/g | <10 CFU/g |
| Count | | | |

| | | |
|---|---|---|
| Enterobacteriaceae | NA | <10 CFU/g |
| count | | |

| | | | |
|---|---|---|---|
| Yeast count | <1000 | CFU/g | <10 CFU/g |
| Mold count | <1000 | CFU/g | <10 CFU/g |

The total aerobic organism plate counts, total Enterobacteriaceae counts, total yeast counts, and total mold counts for non-irradiated raw complete almond allergen substance and 7.5 kGy beta-irradiated almond allergen drug substance, are presented in TABLE 24. Irradiation with 7.5 kGy resulted in significant reduction in the total aerobic plate count.

TABLE 24

| | | |
|---|---|---|
| Almond | | |
| Allergen | Non- | 7.5 kGy Beta |
| Substance | irradiated | Radiation Dose |

| | | | |
|---|---|---|---|
| Total Aerobic Plate | 9,600 | CFU/g | <10 CFU/g |
| Count | | | |
| Enterobacteriaceae | <10 | CFU/g | <10 CFU/g |
| count | | | |
| Yeast count | <10 | CFU/g | <10 CFU/g |
| Mold count | <10 | CFU/g | <10 CFU/g |

The total aerobic organism plate counts, total Enterobacteriaceae counts, total yeast counts, and total mold counts for non-irradiated raw complete peanut allergen substance and 7.5 kGy beta-irradiated peanut allergen drug substance, are presented in TABLE 25. Irradiation with 7.5 kGy resulted in significant reduction in the total aerobic plate count.

TABLE 25

| | | |
|---|---|---|
| Peanut | | |
| Allergen | Non- | 7.5 kGy Beta |
| Substance | irradiated | Radiation Dose |

| | | | |
|---|---|---|---|
| Total Aerobic Plate | 110 | CFU/g | <10 CFU/g |
| Count | | | |

| | | |
|---|---|---|
| Enterobacteriaceae | NA | <10 CFU/g |
| count | | |

| | | | |
|---|---|---|---|
| Yeast count | <10 | CFU/g | <10 CFU/g |
| Mold count | <10 | CFU/g | <10 CFU/g |

The total aerobic organism plate counts, total Enterobacteriaceae counts, total yeast counts, and total mold counts for non-irradiated raw complete sesame allergen substance and 7.5 kGy beta-irradiated sesame allergen drug substance, are presented in TABLE 26. Irradiation with 7.5 kGy resulted in significant reduction in the total aerobic plate count.

TABLE 26

| | | |
|---|---|---|
| Sesame | | |
| Allergen | Non- | 7.5 kGy Beta |
| Substance | irradiated | Radiation Dose |

| | | | |
|---|---|---|---|
| Total Aerobic Plate | 280 | CFU/g | <10 CFU/g |
| Count | | | |

| | | |
|---|---|---|
| Enterobacteriaceae | NA | <10 CFU/g |
| count | | |

| | | | |
|---|---|---|---|
| Yeast count | <10 | CFU/g | <10 CFU/g |
| Mold count | <10 | CFU/g | <10 CFU/g |

The total aerobic organism plate counts, total Enterobacteriaceae counts, total yeast counts, and total mold counts for non-irradiated raw complete soy allergen substance and 7.5 kGy beta-irradiated soy allergen drug substance, are presented in TABLE 27. Irradiation with 7.5 kGy resulted in significant reduction in the total aerobic plate count.

TABLE 27

| | | |
|---|---|---|
| Soy Allergen | Non- | 7.5 kGy Beta |
| Substance | irradiated | Radiation Dose |

| | | | |
|---|---|---|---|
| Total Aerobic Plate | 400 | CFU/g | <10 CFU/g |
| Count | | | |

| | | |
|---|---|---|
| Enterobacteriaceae | NA | <10 CFU/g |
| count | | |

| | | | |
|---|---|---|---|
| Yeast count | 3 | CFU/g | <10 CFU/g |
| Mold count | 3 | CFU/g | <10 CFU/g |

The total aerobic organism plate counts, total Enterohacteriaceae counts, total yeast counts, and total mold counts for non-irradiated raw complete peanut allergen substance and 7.5 kGy beta-irradiated peanut allergen drug substance, are presented in TABLE 28. Irradiation with 7.5 kGy resulted in significant reduction in the total aerobic plate count.

TABLE 28

| | | |
|---|---|---|
| Peanut | | |
| Allergen | Non- | 7.5 kGy Beta |
| Substance | irradiated | Radiation Dose |

| | | | |
|---|---|---|---|
| Total Aerobic Plate | 110 | CFU/g | <10 CFU/g |
| Count | | | |

| | | |
|---|---|---|
| Enterobacteriaceae | NA | <10 CFU/g |
| count | | |

| | | | |
|---|---|---|---|
| Yeast count | <10 | CFU/g | <10 CFU/g |
| Mold count | <10 | CFU/g | <10 CFU/g |

TABLE 29 shows the total aerobic organism plate counts, total E. faecium counts, total yeast counts, and total mold counts for non-irradiated raw complete hen's egg allergen substance, 7.5 kGy beta-irradiated hen's egg allergen drug substance, and 15 kGy beta-irradiated hen's egg allergen drug substance. Irradiation with 7.5 kGy and 15 kGy resulted in significant reduction in the total aerobic plate count and mold count.

TABLE 29

| Hen's Egg | | | | |
|---|---|---|---|---|
| Allergen | Non- | | 7.5 kGy Beta | 15 kGy Beta |
| Substance | irradiated | | Radiation Dose | Radiation Dose |
| Total Aerobic | 720 | CFU/g | <10 CFU/g | <10 CFU/g |
| Plate Count | | | | |
| Enterobacteriaceae | <10 | CFU/g | <10 CFU/g | <10 CFU/g |
| count | | | | |
| Yeast count | <10 | CFU/g | <10 CFU/g | <10 CFU/g |
| Mold count | 10 | CFU/g | <10 CFU/g | <10 CFU/g |

The total aerobic organism plate counts, total Enterobacteriaceae counts, total yeast counts, and total mold counts for non-irradiated raw complete bovine milk allergen substance, 7.5 kGy beta-irradiated bovine milk allergen drug substance, and 15 kGy beta-irradiated bovine milk allergen drug substance are presented in TABLE 30. Irradiation with 7.5 kGy resulted in significant reduction in the total aerobic plate count.

TABLE 30

| Bovine Milk | | | | | |
|---|---|---|---|---|---|
| Allergen | Non- | | 7.5 kGy Beta | 15 kGy Beta | |
| Substance | irradiated | | Radiation Dose | Radiation Dose | |
| Total Aerobic Plate | 710 | CFU/g | <10 CFU/g | 10 | CFU/g |
| Count | | | | | |
| Enterobacteriaceae | <10 | CFU/g | <10 CFU/g | <10 | CFU/g |
| count | | | | | |
| Yeast count | <10 | CFU/g | <10 CFU/g | <10 | CFU/g |
| Mold count | <10 | CFU/g | <10 CFU/g | 10 | CFU/g |

The total aerobic organism plate counts, total Enterobacteriaceae counts, total yeast counts, and total mold counts for non-irradiated raw complete wheat allergen substance, and 7.5 kGy beta-irradiated wheat allergen drug substance are presented in TABLE 31. Irradiation with 7.5 kGy resulted in significant reduction in the total aerobic plate count.

TABLE 31

| | Non- | 7.5 kGy Beta |
|---|---|---|
| Wheat Allergen Substance | irradiated | Radiation Dose |
| Total Aerobic Plate Count | 200 CFU/g | <10 CFU/g |
| Enterobacteriaceae count | NA | <10 CFU/g |
| Yeast count | <10 CFU/g | <10 CFU/g |
| Mold count | <10 CFU/g | <10 CFU/g |

The total aerobic organism plate counts, total Enterobacteriaceae counts, total yeast counts, and total mold counts for non-irradiated raw complete cod allergen substance, and 7.5 kGy beta-irradiated cod allergen drug substance are presented in TABLE 32. Irradiation with 7.5 kGy resulted in significant reductions in the total aerobic plate count and Enterobacteriaceae count.

TABLE 32

| | | |
|---|---|---|
| | Non- | 7.5 kGy Beta |
| Cod Allergen Substance | irradiated | Radiation Dose |

| | | | |
|---|---|---|---|
| Total Aerobic Plate Count | 300 | CFU/g | <10 CFU/g |
| Enterobacteriaceae count | 60 | CFU/g | <10 CFU/g |
| Yeast count | <10 | CFU/g | <10 CFU/g |
| Mold count | <10 | CFU/g | <10 CFU/g |

The total aerobic organism plate counts, total Enterobacteriaceae counts, total yeast counts, and total mold counts for non-irradiated raw complete shrimp allergen substance, and 7.5 kGy beta-irradiated shrimp allergen drug substance are presented in TABLE 33. Irradiation with 7.5 kGy resulted in a reduction in the total aerobic plate count.

TABLE 33

| | | |
|---|---|---|
| | Non- | 7.5 kGy Beta |
| Shrimp Allergen Substance | irradiated | Radiation Dose |

| | | | |
|---|---|---|---|
| Total Aerobic Plate Count | 10 | CFU/g | <10 CFU/g |

| | | |
|---|---|---|
| Enterobacteriaceae count | NA | <10 CFU/g |

| | | | |
|---|---|---|---|
| Yeast count | <10 | CFU/g | <10 CFU/g |
| Mold count | <10 | CFU/g | <10 CFU/g |

Separate samples can also be inoculated with a population of an indicator vegetative organism, Enterococcus faecium NRRL B-2354, prior to undergoing beta radiation treatment, and the total E. faecium can be measured before and after irradiation for inoculated non-irradiated and irradiated milk allergen substance and egg allergen substance samples. For samples that are not inoculated with E. faecium, the limit of detection for the method is 1 CFU/g for a 1:10 dilution. For samples inoculated with E. faecium, the limit of detection is set to the first serial dilution at which no background microflora growth is observed in the samples not inoculated with E. faecium.

### Example 6

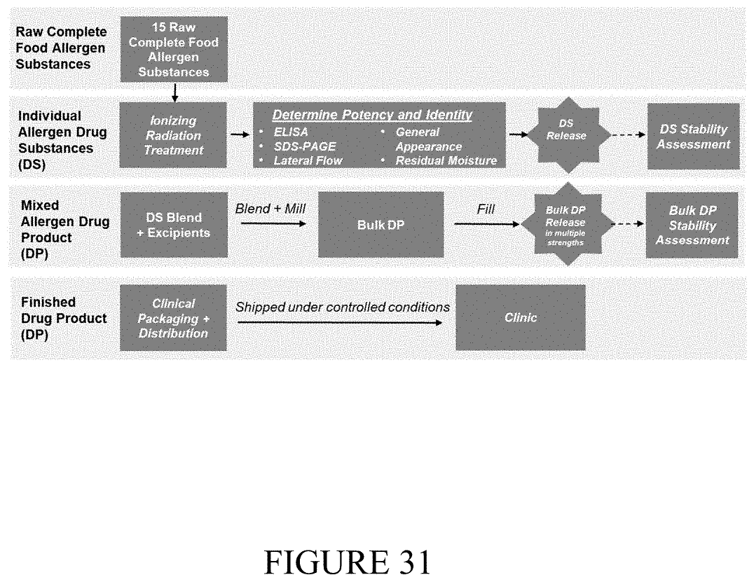

As shown in the manufacturing workflow schematic of FIG. 31, raw complete food allergen substances can be processed to a finished drug product for clinical packaging and distribution. In brief, 15 complete food allergen substances are subjected to ionizing radiation treatment to produce individual allergen drug substances, which are then analyzed to determine potency and identity. Individual allergen drug substances and blended and milled with excipients to form a bulk mixed allergen drug product. Bulk mixed allergen drug products are packaged to form the finished mixed allergen drug product for clinical distribution under controlled shipping conditions. After production of individual allergen drug substances and bulk mixed allergen drug product, samples are released for stability assessments.

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

What is claimed is:

1. A method of making a sterile mixed allergen drug product with known potency and identity that is substantially free of replication viable organisms, the method comprising:
separately irradiating each of 2 to 20 different raw complete food allergen substances, wherein irradiating comprises applying ionizing radiation to each individual raw complete food allergen substance, thereby producing 2 to 20 individual allergen drug substances each substantially free of replication viable organisms, and wherein each individual allergen drug substance retains substantially intact, allergenic proteins; and
blending the 2 to 20 individual allergen drug substances together, thereby obtaining the mixed allergen drug product.

2. The method of claim 1, wherein the individual raw complete food allergen substances are selected from the group consisting of hazelnut, cashew, pistachio, walnut, pecan , almond, peanut, sesame, soy, hen's egg, bovine milk, wheat, salmon, cod, and shrimp.

3. The method of claim 1 or 2, wherein blending further comprises blending the 2 to 20 individual allergen drug substances with one or more bulking agents and/or pharmaceutically acceptable excipients.

4. The method of any one of claims 1 to 3, wherein the applying ionizing radiation is applying beta radiation, gamma radiation, alpha radiation, X radiation, or a combination thereof.

5. The method of any one of claims 1 to 4, wherein the applying ionizing radiation is applying one or more doses of radiation of about 0.15 kilograys to about 30 kilograys.

6. The method of any one of claims 1 to 5, wherein applying ionizing radiation causes about a 0.25 to about 0.5° C. per kilogray dose increase in temperature in the raw complete food allergen sub stance.

7. The method of any one of claims 1 to 6, wherein the ionizing radiation is produced by a particle emitter having an energy of about 0.5 MeV to about 10 MeV.

8. The method of any one of claims 4 to 7, wherein the beta radiation is single or double sided.

9. The method of any one of claims 4 to 7, wherein the gamma radiation is produced by cobalt-60 or cesium-137.

10. The method of any one of claims 4 to 7, wherein the X radiation is produced using tungsten or tantalum.

11. The method of any one of claims 1 to 4, wherein applying ionization radiation comprises applying a dose of beta radiation at 5.0 kilograys, 7.5 kilograys, 15 kilograys, or more.

12. The method of claim 11, wherein applying the dose of beta radiation occurs more than once.

13. The method of any one of claims 1 to 12, wherein the method further comprises milling the mixed allergen drug product to obtain a substantially consistent particle size.

14. The method of any one of claims 1 to 13, wherein the method further comprises milling one or more than one of the raw complete food allergen substances.

15. The method of any one of claims 1 to 14, wherein the method further comprises milling one or more than one of the individual allergen drug substances.

16. The method of any one of claims 1 to 15, further comprising independently packaging each of the 2 to 20 raw complete food allergen substances into separate irradiation compatible packaging before irradiating.

17. The method of any one of claims 1 to 16, wherein each of the 2 to 20 individual allergen drug substances has less than about 1000 CFU/g of aerobic bacterial organisms.

18. The method of any one of claims 1 to 17, wherein each of the 2 to 20 individual allergen drug substances has less than about 100 CFU/g of aerobic bacterial organisms.

19. The method of any one of claims 1 to 18, wherein each of the 2 to 20 individual allergen drug substances has less than about 10 CFU/g of aerobic bacterial organisms.

20. The method of any one of claims 1 to 19, wherein each of the 2 to 20 individual allergen drug substances has less than about 10 CFU/g of Enterobacteriaceae.

21. The method of any one of claims 1 to 20, wherein each of the 2 to 20 individual allergen drug substances has less than about 100 CFU/g of yeast.

22. The method of any one of claims 1 to 21, wherein each of the 2 to 20 individual allergen drug substances has less than about 10 CFU/g of yeast.

23. The method of any one of claims 1 to 22, wherein each of the 2 to 20 individual allergen drug substances has less than about 100 CFU/g of mold.

24. The method of any one of claims 1 to 23, wherein each of the 2 to 20 individual allergen drug substances has less than about 10 CFU/g of mold.

25. The method of any one of claims 1 to 24, wherein each of the 2 to 20 individual allergen drug substances has about 1% to about 10% moisture.

26. The method of any one of claims 1 to 25, wherein at least one of the 2 to 20 individual allergen drug substances has about 4% to about 7% moisture.

27. The method of any one of claims 1 to 26, wherein each of the 2 to 20 individual allergen drug substances has about 0.2 to about 0.6 water activity.

28. The method of any one of claims 1 to 27, wherein each individual allergen drug substance has substantially the same protein integrity as compared to a corresponding raw complete food allergen substance.

29. The method of any one of claims 1 to 28, wherein the protein integrity of each individual allergen drug substance is determined by SDS-PAGE.

30. The method of any one of claims 1 to 29, wherein the protein content/potency and/or identity of each individual allergen drug substance is tested by ELISA.

31. The method of any one of claims 1 to 30, wherein the protein content/potency and/or identity of each raw complete food allergen substance is tested by ELISA.

32. The method of any one of claims 1 to 31, wherein each individual allergen drug substance has a substantially similar allergen effect upon administration to a patient as administration of the substantially same protein amount of a corresponding raw complete food allergen substance.

33. The method of claim 32, wherein allergen effect is measured by immune response in the patient.

34. The method of any one of claims 1 to 33, wherein the mixed allergen drug product comprises 6 to 20 individual allergen drug substances.

35. The method of any one of claims 1 to 34, wherein the mixed allergen drug product comprises about 0.1 mg to about 500 mg, by protein mass, of each individual allergen drug sub stance.

36. The method of any one of claims 1 to 35, wherein the mixed allergen drug product comprises 15 or 16 individual allergen drug substances, wherein each individual allergen drug substance is present in about a 2:1 to about 1:2 ratio, by protein weight, with another individual allergen drug substance.

37. The method of any one of claims 1 to 36, wherein the mixed allergen drug product comprises substantially equal amounts of individual allergen drug substances by total protein weight.

38. The method of any one of claims 1 to 37, wherein the individual raw complete food allergen substances are selected from the group consisting of hazelnut flour, cashew flour, pistachio flour, walnut flour, pecan flour, almond flour, peanut flour, sesame flour, soy flour, hen's egg powder, bovine milk powder, wheat flour, salmon powder, cod powder, and shrimp powder.

39. The method of any one of claims 1 to 38, wherein the individual allergen drug substances are stable for at least 6 months.

40. The method of any one of claims 1 to 39, wherein the individual allergen drug substances are stable for at least one year.

41. The method of any one of claims 1 to 40, wherein the mixed allergen drug product is stable for at least 6 months.

42. The method of any one of claims 1 to 41, wherein the mixed allergen drug product is stable for at least 1 year.

43. A method of making a sterile mixed allergen drug product substantially free of replication viable organisms, the method comprising:
providing 2 to 20 individual irradiated allergen drug substances each substantially free of replication viable organisms and wherein each individual allergen drug substance retains substantially intact, allergenic proteins; and
blending the 2 to 20 individual allergen drug substances together, thereby obtaining the mixed allergen drug product.

44. A method of making a mixed allergen drug product substantially free of replication viable organisms, the method comprising:
providing 6 to 20 different raw complete food allergen substances;
blending the 6 to 20 different raw complete food allergen substances to produce a bulk substance; and
irradiating the bulk substance with ionizing radiation,
thereby obtaining the mixed allergen drug product.

45. A mixed allergen drug product that is substantially free of replication viable organisms prepared by the method of any one of claims 1 to 44.

46. The mixed allergen drug product of claim 45, wherein the mixed allergen drug product is for oral immunotherapeutic treatment of food allergy in a child or adult.

47. The mixed allergen drug product of claim 46, wherein mixed allergen drug product is for mixture with a food to which the child or adult is not allergic

48. A method of making a sterile allergen drug product substantially free of replication viable organisms, the method comprising:
irradiating a raw complete food allergen substance, wherein irradiating comprises applying ionizing radiation to the raw complete food allergen substance, thereby producing an individual allergen drug substance substantially free of replication viable organisms and wherein the allergen drug substance retains substantially intact, allergenic proteins.

49. The method of claim 48, wherein the raw complete food allergen substance is selected from the group consisting of hazelnut, cashew, pistachio, walnut, pecan , almond, peanut, sesame, soy, hen's egg, bovine milk, wheat, salmon, cod, and shrimp.
